SEQUENCE LISTING (26 sequences)

SEQ ID NO: 1 - DNA, 852 bp, Lemna turionifera
  source 1..852: mol_type = other DNA
atggggagagcgccttgctgcgagaaggacgggctgaagaaggggagatggactgcggaggaagacaagattctggcgaattatattcgagtcaatggagaaggttcatggagatcgctgccgaagaatgcaggacttcttcgatgcgggaagagttgcaggctgagatggataaactacctcagatctgacttgaagagaggcaacatttctccggaagaggaggagatgatcgtccggcttcatacttctctcggaaacagatggtctctgatcgccgcccagctgcctgggaggacggataacgagatcaagaactactggaactcccatctgagccgtaggattcaccggtttaagagactcgacgggggcgatggctccacggtgatcgttgacctgagcaagatcgtcggaattccgtgccggcggcgaggaggaagaaaaaagatttcgccaatccaaagcgttgagcccgagagtaaaaaagtcgaccacaaggagatgctgatggagacggcgacgaacgagttgctgagcccgagttgggagagcggcgggtcgatgagctcgtgtacgacgagcgaggagaggaaagacgagccagttccgtcaaaggaaaaacagctctacgctcatggagaagagtgcaacagctctgcaacaacggagggagaaatgcttgacgtggactgggaagccatggctacgaagctttgggataatgacgagatggaggaaatgtggccatggctctgggataatagctcgcatggattccagctcaagtctcatctgcagccgccggagggtgggcaagaggagacagatctcggggaatggatcctctga

SEQ ID NO: 2 - protein, 283 aa, Lemna turionifera
MGRAPCCEKDGLKKGRWTAEEDKILANYIRVNGEGSWRSLPKNAGLLRCGKSCRLRWINYLRSDLKRGNISPEEEEMIVRLHTSLGNRWSLIAAQLPGRTDNEIKNYWNSHLSRRIHRFKRLDGGDGSTVIVDLSKIVGIPCRRRGGRKKISPIQSVEPESKKVDHKEMLMETATNELLSPSWESGGSMSSCTTSEERKDEPVPSKEKQLYAHGEECNSSATTEGEMLDVDWEAMATKLWDNDEMEEMWPWLWDNSSHGFQLKSHLQPPEGGQEETDLGEWIL

SEQ ID NO: 3 - DNA, 18 bp, synthetic construct
  source 1..18: mol_type = other DNA
atggggagagccttgctg

SEQ ID NO: 4 - DNA, 21 bp, synthetic construct
  source 1..21: mol_type = other DNA
tcagaggatcccccgagatct

SEQ ID NO: 5 - DNA, 861 bp, Landoltia punctata
  source 1..861: mol_type = other DNA
atggggagggcgccgtgttgtgagaaagtgggaatgaagaagggaagatggacggcagaggaggacgagatactggcgaactacataaagaagaacggagaaggttcctggagatcgttgccgaagaaagcaggtctctcgaggtgcggaaagagctgcagacttcgctggataaactatctgagaagtgatctcaagaggggaaacatcactttggaggaagaggagaccatcctgaagcttcacacttctctcggcaacagatggtccctgatagccgcccaccttcccggaagaacggacaacgagataaagaaccactggaacgcgcacctcagccgccgaatccacgtcttcgggcggggtgaccccgtgatcctggatctcggacggatggctcacgggagacggcgggggcatgaagccgctgccgaacccacctccacgaagagaccaggcggcggcggtgccgctgcggtgaaaatcgccggacaagaagagctattggtaagagagaaacccctgatcgatgggggaatcttggaagagtttgagatccttcccggcgagttctgggagatgattgatgatatcggcgggaatatgctctccgcaagcgcgacgtccaccccggcgacgagcggggaggttgactcgacgggtgctggaacgggagacttcggcgagctctcgtgcggcagcagcggtggcaagatgttcggcgagcagtcggatgaggagtgggaaatcatggcggcacagctatgggacgggaccgaaaaagcgtcgccgaggcgatcggacgatttcatttacgacgacggcgccctcgactggttcatctctcacgtcctgtcgtaa

SEQ ID NO: 6 - protein, 286 aa, Landoltia punctata
MGRAPCCEKVGMKKGRWTAEEDEILANYIKKNGEGSWRSLPKKAGLSRCGKSCRLRWINYLRSDLKRGNITLEEEETILKLHTSLGNRWSLIAAHLPGRTDNEIKNHWNAHLSRRIHVFGRGDPVILDLGRMAHGRRRGHEAAAEPTSTKRPGGGGAAAVKIAGQEELLVREKPLIDGGILEEFEILPGEFWEMIDDIGGNMLSASATSTPATSGEVDSTGAGTGDFGELSCGSSGGKMFGEQSDEEWEIMAAQLWDGTEKASPRRSDDFIYDDGALDWFISHVLS

SEQ ID NO: 7 - DNA, 25 bp, synthetic construct
  source 1..25: mol_type = other DNA
atggggagggcgccgtgttgtgaga

SEQ ID NO: 8 - DNA, 25 bp, synthetic construct
  source 1..25: mol_type = other DNA
ttacgacaggacgtgagagatgaac

SEQ ID NO: 9 - DNA, 23 bp, synthetic construct
  source 1..23: mol_type = other DNA
gtccggcttcatacttctctcgg

SEQ ID NO: 10 - DNA, 24 bp, synthetic construct
  source 1..24: mol_type = other DNA
gtccggcttcatacttcttctcgg

SEQ ID NO: 11 - DNA, 23 bp, synthetic construct
  source 1..23: mol_type = other DNA
cgacgggggcgatggctccacgg

SEQ ID NO: 12 - DNA, 24 bp, synthetic construct
  source 1..24: mol_type = other DNA
cgacgggggcgatggcttccacgg

SEQ ID NO: 13 - protein, 343 aa, Arabidopsis thaliana
MGRAPCCEKVGIKKGRWTAEEDRTLSDYIQSNGEGSWRSLPKNAGLKRCGKSCRLRWINYLRSDIKRGNITPEEEDVIVKLHSTLGTRWSTIASNLPGRTDNEIKNYWNSHLSRKLHGYFRKPTVANTVENAPPPPKRRPGRTSRSAMKPKFILNPKNHKTPNSFKANKSDIVLPTTTIENGEGDKEDALMVLSSSSLSGAEEPGLGPCGYGDDGDCNPSINGDDGALCLNDDIFDSCFLLDDSHAVHVSSCESNNVKNSEPYGGMSVGHKNIETMADDFVDWDFVWREGQTLWDEKEDLDSVLSRLLDGEEMESEIRQRDSNDFGEPLDIDEENKMAAWLLS

SEQ ID NO: 14 - protein, 371 aa, Arabidopsis thaliana
MGRAPCCEKVGIKRGRWTAEEDQILSNYIQSNGEGSWRSLPKNAGLKRCGKSCRLRWINYLRSDLKRGNITPEEEELVVKLHSTLGNRWSLIAGHLPGRTDNEIKNYWNSHLSRKLHNFIRKPSISQDVSAVIMTNASSAPPPPQAKRRLGRTSRSAMKPKIHRTKTRKTKKTSAPPEPNADVAGADKEALMVESSGAEAELGRPCDYYGDDCNKNLMSINGDNGVLTFDDDIIDLLLDESDPGHLYTNTTCGGDGELHNIRDSEGARGFSDTWNQGNLDCLLQSCPSVESFLNYDHQVNDASTDEFIDWDCVWQEGSDNNLWHEKENPDSMVSWLLDGDDEATIGNSNCENFGEPLDHDDESALVAWLLS

SEQ ID NO: 15 - protein, 417 aa, Nicotiana tabacum
MGRAPCCEKVGLKRGRWTAEEDEILTKYIQTNGEGSWRSLPKNAGLLRCGKSCRLRWINYLRSDLRRGNITSEEEDIIIKLHATLGNRWSLIAAHLPGRTDNEIKNYWNSHLSRKVESLRIPSDEKLPQAVVDLAKKGTLNPIKCRVGKTSRPSVKKNKTFKKSSSSLSEPKQPKESSEPLNSIVPMPSTPNMEKEALSSTISSWLEGNNVMDSMQEEVANVAAPNPWSGSREAQSSLSSDSGMEWLEEIMPMVIADQDMDPNEFILTYLDNGQGESPEKVTNEAENNCGTTNRINERNNKDHVNKMVSSENTQLESSPESETVSINILKDVQENSNESKLLMEDSTVEWDWQEIADDMREVWSWEETGQDNMLINHSWPLWDNTGTEFTNEITVEMDSVLHSENPNHSALVAWLLS

SEQ ID NO: 16 - protein, 364 aa, Setaria italica
MGRAPCCEKVGLKRGRWTAEEDEILANYIAKHGEGSWRSLPKNAGLLRCGKSCRLRWINYLRADVKRGNISKEEEDVIIKLHATLGNRWSLIASHLPGRTDNEIKNYWNSHLSRQIHTYRRTYTAGPDTTITIDISKLHSAEKRRGGRTPGRSPKRATTSSGGGGGGSNKAKSKELPDPDSKKPEPESGEAKVASGPVAAAASAASSPRHSDGARSAVVDPDQNQPDSSSGAGGGSTPEGPCSEDTTGPLAALDPMEFGGLWEAESEMELLLSSGGVEAGPDPLTGFVDAVGEAGQVDDLLDMDWDGFAAHIWGDHPTAQVQQQNDDDQSTLLQPDGPQAAAGCNEHQEDELESFATWLLSDSF

SEQ ID NO: 17 - protein, 336 aa, Zea mays
MGRAPCCEKVGLKRGRWTAEEDQLLANYIAEHGEGSWRSLPKNAGLLRCGKSCRLRWINYLRADVKRGNISKEEEDIIIKLHATLGNRWSLIASHLPGRTDNEIKNYWNSHLSRQIHTYRRKYTAGPDDTAIAIDMSKLQSADRRRGGRTPGRPPKASASRTKQADADQPGGEAKGPAAAASSPRHSDVVNPGPNQPNSSSGSTGTAEEEGPSSEDASGPWVLEPIELGDLVWGEADSEMDALMPIGPGGHDSAALEGLGAVGCEAQVDDLFDMDWDGFAAHLWGGPEQDEHSAQLRQAAEPLEVAAAAAAAATAARTPDDRELEAFETWLLSDSF

SEQ ID NO: 18 - protein, 344 aa, Spirodela polyrrhiza
MGRAPCCQKEGLKKGRWTSEEDEILVNYIKANGEGSWRTLPKNAGLLRCGKSCRLRWINYLRSDLKRGNITPEEEEVIIKLHISLGNRWSLIAAQLPGRTDNEIKNHWNSHLSRRIHRLRRLDGDDGPAVIMDLGKIVGIPCRRRGGRMSRSAMKKESTVGTVDKGQHGQGGPDGESEKTAQPTCGRPVEFPSKKVNPEALPACEPPETDPSLPTEFWETASELLSPSWERGDGGWSAASSFIEQRESGPSMSSCTTSEERKDGTMPVVAHQPYAQADECNSTGTTGGGWLDVELEAIATKLWDEAGEAWPWQWEDASVVDLQSHQRPTDCCEYERGALESWLL

SEQ ID NO: 19 - DNA, 23 bp, synthetic construct
  source 1..23: mol_type = other DNA
ggtgagatgcaccggatctccgg

SEQ ID NO: 20 - DNA, 24 bp, synthetic construct
  source 1..24: mol_type = other DNA
ggtgagatgcaccggatcctccgg

SEQ ID NO: 21 - DNA, 23 bp, synthetic construct
  source 1..23: mol_type = other DNA
catattgttcgggtccccaatgg

SEQ ID NO: 22 - DNA, 24 bp, synthetic construct
  source 1..24: mol_type = other DNA
catattgttcgggtcccacaatgg

SEQ ID NO: 23 - DNA, 10 bp, synthetic construct
  source 1..10: mol_type = other DNA
ctacctaatt

SEQ ID NO: 24 - DNA, 15 bp, synthetic construct
  source 1..15: mol_type = other DNA
atcctctacctaatt

SEQ ID NO: 25 - DNA, 10 bp, synthetic construct
  source 1..10: mol_type = other DNA
ctctacctaa

SEQ ID NO: 26 - DNA, 11 bp, synthetic construct
  source 1..11: mol_type = other DNA
cctctacctaa

(19) United States
(12) Patent Application Publication
(10) Pub. No.: US 2026/0193666 A1
(43) Pub. Date: Jul. 9, 2026

(54) ENHANCED PRODUCTION OF FLAVONOIDS

(71) Applicant: QINGDAO INST BIOENERGY & BIOPROCESS TECH CAS

(72) Inventors: Shengjun LI, Shandong (CN); Ruibo HU, Shandong (CN); Hua XU, Shandong (CN); Shumin WANG, Shandong (CN); Guo HE, Shandong (CN)

(73) Assignee: QINGDAO INST BIOENERGY & BIOPROCESS TECH CAS, Shandong (CN)

(21) Appl. No.: 19/132,110

(22) PCT Filed: Nov. 22, 2023
(86) PCT No.: PCT/EP2023/082715

§ 371 Date: May 22, 2025

(30) Foreign Application Priority Data
Nov. 25, 2022 (CN) 202211492405.2

(51) Int. Cl. C12N 15/82; C07K 14/415

(52) U.S. Cl. CPC C12N 15/8243; C07K 14/415; C12N 15/8205

(57) ABSTRACT

The present invention discloses the application of Duckweed Pericarp Color1 (P1) protein homologues (P1-Ls) such as LtP1-L or LeP1-L in regulating the biosynthesis of orientin and/or isoorientin in duckweed or other plants. Specifically, P1-L was introduced into the duckweed genome and overexpressed. The transgenic duckweed generates higher content of orientin and/or isoorientin than wild-type duckweed. The present invention also discloses a method for biosynthesizing orientin and/or isoorientin: P1-L was introduced into the duckweed genome, P1-L overexpressing duckweed with higher content of orientin and/or isoorientin than wild-type plants; Cultivation of the transgenic duckweed and extraction of orientin and/or isoorientin. The present invention greatly reduces the production costs of orientin and isoorientin, and has broad application prospects and high economic value.

### CROSS-REFERENCE TO RELATED APPLICATIONS

This patent application claims the benefit of priority of CN202211492405 filed 25 Nov. 2022 and which is herein incorporated in its entirety.

### BACKGROUND TO THE INVENTION

Flavonoids are a major class of secondary metabolites in plants, which accumulate in different plant organs. Based on the variations in the C6-C3-C6 carbon skeleton, thousands of flavonoid compounds are generally divided into several subgroups, including flavone, flavonol, flavanone, flavanonol, flavanol, isoflavone and anthocyanidin (Ku et al., 2020).

As one class of flavones, C-glycosylated flavones (CGFs) comprise at least one sugar moiety, which is bound to the aglycone scaffold through a direct C—C glycosidic bond.

Uke other flavonoids, CGFs have been used as valuable nutraceuticals and are beneficial for human health associated with their anti-nociception, anti-Alzheimer's disease, anti-diabetic, and neuroprotection (Choi et al., 2014; Courts and Williamson, 2015; Min et al., 2015; Zhu et al., 2016; Sun et al., 2020). However, the C—C linkage formed by C-glycosylation is more stable facing acid and glycoside hydrolases compared with 0-C bond (Brazier-Hicks et al., 2009; Ferreyra et al., 2013). Moreover, CGFs are more rapidly absorbed by human gastrointestinal system than the corresponding 0-glycosides (Courts and Williamson, 2015). The potential human benefits of CGFs have attracted more and more attention due to their being more orally bioavailable (Vanegas et al., 2018).

Generally, the first committed step in the biosynthesis of flavonoid is catalyzed by chalcone synthase (CHS) and then a chalcone is transformed into a flavanone under the catalysis of chalcone isomerase (CHI), thus forming the basic C6-C3-C6 carbon flavonoid skeleton of flavonoid. Subsequently, a battery of hydroxylases, reductases and dioxygenases modify the basic carbon skeleton and produce different flavonoid subclasses (Martens et al., 2010).

For CGFs, two distinct biosynthetic pathways have been reported so far. UF6CGT1 identified from Gentiana triflora catalysed the direct C-glycosylation at the C6 position of a flavone skeleton (Sasaki et al., 2015). In some cereals, the C-glycoside is usually transferred to an open-ring form of 2-hydroxylated intermediate produced by a flavanone 2-hydroxylase (F2H) (Du et al., 2010), and the C-glycosylation is catalysed by C-glycosyltransferases (CGTs) exclusively from UGT708 subfamily resulting in the flavone-6-C or -8-C-glucosides (Sun et al., 2020).

Flavonoid biosynthesis is generally controlled by some specific R2R3-MYB transcription factors (Uu et al., 2015). AtMYB11, AtMYB12 and AtMYB111 which independently activate the expression of CHS, CHI, flavanone 3-hydroxylase (F3H), and flavonol synthase (FLS) to promote biosynthesis of flavonol (Stracke et al., 2007). The isoflavone content in soybean roots increased significantly by overexpressing an R2R3-MYB transcription factor GmMYB29, which was capable of activating the transcription of isoflavone synthetase 2 (IFS2) and chalcone synthase 8 (CHS8) (Chu et al., 2017). Pericarp Color1 (P1) protein is responsible for the biosynthesis of CGFs in maize via directly up-regulating ZmF2H and ZmCGT (Morohashi et al., 2012).

C-glycosylated flavones are widespread in terrestrial plants, including rice, maize, bamboo and flax (Brazier-Hicks et al., 2009; Casas et al., 2014; Czemplik et al., 2016; Sun et al., 2020).

At present, flavonoids are mainly extracted from natural plant resources through various methods such as traditional extraction, enzymatic extraction, ultrasound assisted extraction, or synthesis through effective chemical means. The above methods have their own advantages and disadvantages, but they all have shortcomings such as low extraction or synthesis efficiency, high cost, and susceptibility to secondary pollution.

### DISCLOSURE OF THE INVENTION

The inventors cloned the P1-L genes from Lemna turionifera and Landoltia punctata, which were named LtP1-L and LeP1-L, respectively. The genes are functionally related sharing over 40% sequence identity.

The present invention relates, inter alia, to the application of Pericarp Color1 (P1) protein homologues from duckweed (LtP1-L and LeP1-L) in regulating the biosynthesis of orientin and isoorientin in plants such as duckweed, and specifically relates to the application of overexpressing LtP1-L and LeP1-L) in increasing the content of orientin and isoorientin, belonging to the field of genetic engineering technology.

The present inventors found that overexpression of LtP1-L and LeP1-L can effectively increase the content of orientin and isoorientin

This was demonstrated, by way of exemplification only, with a duckweed (Lemna turionifera). An aquatic plant, duckweed is also a potential natural source of CGFs (Pagliuso et al., 2020). In addition to the feedstock for bioenergy and fodder (Yu et al., 2014; Sonta et al., 2019), duckweeds have been used as a traditional herbal medicine to cure urticaria, acute nephritis, and influenza, of which Spirodela polyrrhiza, Wolffiella caudata, Wolfia borealis, and Landoltia punctata display higher medicinal activity (Ren et al., 2016; Pagliuso et al., 2020). The present disclosure is believed to be the first demonstrated use of genetic engineering technology to biosynthesis flavonoids in duckweed.

As explained in the Examples hereinafter, overexpression of LtP1-L and LeP1-L) can increase the content of flavonoids such as orientin and isoorientin in duckweed, and these two types of flavonoids can be released in large amounts in the aqueous solution of duckweed cultivation. The content of orientin and isoorientin in cultured aqueous solution is significantly increased, with higher purity and fewer impurities. After simple treatment, genetically engineered duckweed or its hydroponic solution can be applied to industrial processes such as food industry, health products, cosmetics, and feed production.

More specifically, an R2R3-MYB transcription factor LtP1L was cloned from Lemna turionifera, and was shown to be a nuclear localization protein with transcriptional activation activity. LtP1L activated flavonoid biosynthesis, and dramatically increased the content of two main CGFs (i.e., orientin and isoorientin) in L. turionifera. LtP1L and, in addition, regulated the biosynthesis of phenylalanine, which is beneficial for directing metabolic flux into CGFs. Meanwhile, LtP1L directly activated the expression of LtABCC4 involved in the transport of CGF to the vacuole, suggesting that, in L. turionifera, the biosynthesis and transportation of CGFs were cooperatively controlled by a single MYB transcription factor. Interestingly, cultivation of transgenic duckweed with only 2% sucrose significantly increased the yield of CGFs.

The methods of the present invention can greatly reduce the production cost of orientin and isoorientin, and have broad application prospects and potential high economic value.

The present invention is implemented through the following technical solutions:

In one aspect there is provided a method for producing a plant with modified flavonoid biosynthesis, the method comprising altering the expression, or activity, of a P1-L such as LtP1-L or LeP1-L protein in the plant. Typically the expression is altered by use of an LtP1-L or LeP1-L) polynucleotide encoding the LtP1-L or LeP1-L protein, or by targeting endogenous LtP1-L or LeP1-L polynucleotide encoding the LtP1-L or LeP1-L protein. Both approaches are described herein.

“Modified” in this context means as compared to a corresponding plant in which the expression, or activity, of LtP1-L or LeP1-L protein has not been modified e.g. a wild-type plant.

In one embodiment the flavonoids are C-glycosylated flavones.

In one embodiment the flavonoids are orientin and/or isoorientin.

In one embodiment the method comprises increasing the expression of the LtP1-L or LeP1-L protein in the plant.

In one embodiment the method causes:
(i) increased biosynthesis of phenylalanine and/or p-coumaroyl CoA;
(ii) activating of the expression of LtABCC4 e.g. to modify the transport of orientin and/or isoorientin to the vacuole.

In one embodiment the method causes an increase the content of total flavonoids in the plant by about 2, 2.5, or 3 times compared to the corresponding (e.g. wild type) plant

In one embodiment the method causes an increase in the content of orientin and isoorientin by about 5, 6, 7, 8, 9, or 10 or more times compared to the wild type plant

In one embodiment the LtP1-L protein is a variant of native LtP1-L e.g. has a sequence with at least 70% identity to SEQ ID NO:2.

In one embodiment the LeP1-L protein is a variant of native LeP1-L e.g. has a sequence with at least 70% identity to SEQ ID NO:6.

In one embodiment the LtP1-L or LeP1-L protein comprises conserved R2 and R3 domains. Such domains are as show in FIG. 2a.

In one embodiment the LtP1-L protein comprises a partial SG7-2 motif [K/R][R/x][R/K]xGR and a partial SG7 motif (see FIG. 2a).

In one embodiment the LeP1-L protein comprises no SG7-2 motif and a partial SG7 motif (see FIG. 2a).

LtP1-L or LeP1-L protein of the present invention has the biological and functional activities described herein e.g. ability of modify flavonoid biosynthesis in a plant when introduced therein (e.g. increase orientin and/or isoorientin production). Other activities may be the ability to increase biosynthesis of phenylalanine and/or p-coumaroyl CoA or activating of the expression of LtABCC4 e.g. by binding the promoter of LtABCC4.

LtP1-L protein may be a polypeptide fragment of SEQ ID No. 2, preferably comprising at least 100 contiguous amino acids, more preferably at least 150 contiguous amino acids, more preferably at least 200, 250, 260, 270 or 280 contiguous amino acids of SEQ ID No. 2.

LeP1-L protein may be a polypeptide fragment of SEQ ID No. 6, preferably comprising at least 100 contiguous amino acids, more preferably at least 150 contiguous amino acids, more preferably at least 200, 250, 260, 270 or 280 contiguous amino acids of SEQ ID No. 2.

LtP1-L protein or LeP1-L protein may be referred to herein as a “polypeptide of the invention” or “P1-L” protein for brevity.

LtP1-L protein may be encoded by a LtP1-L polynucleotide which has at least 70% identity to SEQ ID NO: 1.

LeP1-L protein may be encoded by a LeP1-L polynucleotide which has at least 70% identity to SEQ ID NO: 5.

Such a polynucleotide may be referred to herein as a “polynucleotide of the invention” for brevity.

LtP1-L protein may be encoded by LtP1-L polynucleotide having SEQ ID NO: 1

LeP1-L protein may be encoded by LeP1-L polynucleotide having SEQ ID NO: 5

Flavonoids are widely found in various plants, including Rutaceae, Ginkgoaceae, Leguminosae, Lamiaceae and Compositae.

Duckweed (Lemnaceae) is a kind of higher aquatic flowering plant that lives by floating, and is the smallest flowering plant in the world. Lemnaceae consists of 5 genera: Spirodela, Landoltia, Lemna, Wolffia, and Wolfella.

As a non food aquatic plant, duckweed has the advantages of fast growth, easy reproduction, low production cost, and can achieve rapid accumulation of starch and protein content. Wild-type duckweed contains flavonoids such as orientin and isoorientin, but their content is very low, for example an orientin content of about 0.38 mg/g dry weight and isoorientin content of about 0.80 mg/g dry weight.

Previously duckweed studies have mainly focused on the response of flavonoid metabolism to certain environmental conditions, including nutrient starvation, heavy metal stress, uniconazole treatment, and photosynthetic redox imbalance (Akhtar et al., 2010; Huang et al., 2014; Guo et al., 2017; Tao et al., 2017). Several heterologous peptides and proteins have been successfully expressed in different duckweed species, such as aprotinin, hirudin, and M2 matrix protein (Rival et al., 2008; Firsov et al., 2018; Khvatkov et al., 2021).

However, the biosynthesis pathway of CGFs generally has been poorly understood, and this likewise applied to its corresponding regulation mechanism in plants such as duckweed.

In one embodiment the plant utilised in the present invention is selected from a species of Rutaceae, Ginkgoaceae, Leguminosae, Lamiaceae and Compositae.

In one embodiment the plant is duckweed (Lemna turionifera).

Thus the invention provides in one embodiment the application of LtP1-L in regulating the biosynthesis of orientin and/or isoorientin in duckweed, wherein the nucleotide sequence of LtP1-L is shown in SEQ ID No. 1.

Thus the invention provides in one embodiment the application of LeP1-L in regulating the biosynthesis of orientin and/or isoorientin in duckweed, wherein the nucleotide sequence of LeP1-L is shown in SEQ ID No. 5.

The invention further provides in one embodiment the application of overexpressing LtP1-L or LeP1-L in increasing the content of orientin and/or isoorientin in duckweed.

The invention further provides the application of LtP1-L protein in regulating the biosynthesis of orientin and/or isoorientin in duckweed, wherein the amino acid sequence of LtP1-L is shown in SEQ ID No. 2.

The invention further provides the application of LeP1-L protein in regulating the biosynthesis of orientin and/or isoorientin in duckweed, wherein the amino acid sequence of LeP1-L is shown in SEQ ID No. 6.

As explained herein, methods of the invention may comprise transforming a plant to express the P1-L protein (e.g. LtP1-L or LeP1-L protein) in the plant.

Methods for transforming plant cells, plants and portions thereof with polypeptides are described in Draper et al., 1988, Plant Genetic Transformation and Gene Expression. A Laboratory Manual. Blackwell Sci. Pub. Oxford, p. 365; Potrykus and Spangenburg, 1995, Gene Transfer to Plants. Springer-Verlag, Berlin; and Gelvin et al., 1993, Plant Molecular Biol. Manual. Kluwer Acad. Pub. Dordrecht. A review of transgenic plants, including transformation techniques, is provided in Galun and Breiman, 1997, Transgenic Plants. Imperial College Press, London.

“Transformed” in this context means that the nucleotide sequences of the heterologous nucleic acid alter one or more of the cell's characteristics and hence phenotype e.g. with respect to flavonoid biosynthesis. Such transformation may be transient or stable.

In one embodiment the method comprises transforming the plant with an LtP1-L polynucleotide encoding the LtP1-L protein.

In one embodiment the method comprises transforming the plant with an LeP1-L polynucleotide encoding the LeP1-L protein.

In one embodiment the LtP1-L polynucleotide is operably linked to a heterologous promoter.

In one embodiment the LeP1-L polynucleotide is operably linked to a heterologous promoter.

In one embodiment the method comprises modifying the sequence of an endogenous LtP1-L polynucleotide encoding the LtP1-L protein in the plant e.g. the promoter or ORF of the polynucleotide.

In one embodiment the method comprises modifying the sequence of an endogenous LeP1-L polynucleotide encoding the LeP1-L protein in the plant e.g. the promoter or ORF of the polynucleotide.

A preferred application is to introduce LtP1-L or LeP1-L into the duckweed genome, thereby overexpressing LtP1-L or LeP1-L, resulting in higher content of orientin and/or isoorientin than corresponding duckweed into which the LtP1-L or LeP1-L is not introduced e.g. wild-type.

One transformation technology well known to those skilled in the art utilises a “binary vector” system which includes (a) border sequences which permit the transfer of a desired nucleotide sequence into a plant cell genome; (b) desired nucleotide sequence itself, which will generally comprise an expression cassette of (i) a plant active promoter, operably linked to (ii) the target sequence and\or enhancer as appropriate. The desired nucleotide sequence is situated between the border sequences and is capable of being inserted into a plant genome under appropriate conditions. The binary vector system will generally require other sequence (derived from Agrobacterium tumefaciens) to effect the integration. Generally this may be achieved by use of so called “agro-infiltration” which uses Agrobacterium-mediated transient transformation. Briefly, this technique is based on the property of A. tumefaciens to transfer a portion of its DNA (“T-DNA”) into a host cell where it may become integrated into nuclear DNA. The T-DNA is defined by left and right border sequences which are around 21-23 nucleotides in length. The infiltration may be achieved e.g. by syringe (in leaves) or vacuum (whole plants). In the present invention the border sequences will generally be included around the desired nucleotide sequence (the T-DNA) with the one or more vectors being introduced into the plant material by agro-infiltration.

In one embodiment, transformation in the present methods is mediated by Agrobacterium to infect the callus of duckweed.

Thus a method may include the following steps:

(1) LtP1-L or LeP1-L is inserted into a plant expression vector, and a plant overexpression vector containing LtP1-L or LeP1-L is constructed;
LtP1-L or LeP1-L is obtained through PCR amplification, and the template can be either duckweed genomic DNA or cDNA (obtained by extracting the total RNA of duckweed and then reverse transcription to obtain cDNA). The specific primers used for amplification of LtP1-L can be as follows:

Forward primer P1:
(SEQ ID No. 3)
5′-ATGGGGAGAGCCTTGCTG-3′
Reverse primer P2:
(SEQ ID No. 4)
5′-TCAGAGGATCCCCCGAGATCT-3′;

The specific primers used for amplification of LeP1-L can be as follows:

Forward:
5′ ATGGGGAGGGCGCCGTGTTGTGAGA 3′
Reverse:
5′ TTACGACAGGACGTGAGAGATGAAC 3′

(2) Plant overexpression vector containing LtP1-L or LeP1-L is transformed into A. tumefaciens, and then transfected into duckweed callus.

In one aspect there is provided an isolated LtP1-L or LeP1-L polynucleotide encoding an LtP1-L or LeP1-L protein as defined herein.

In one aspect there is provided a genetic construct comprising a LtP1-L or LeP1-L polynucleotide encoding an LtP1-L protein as defined herein.

In one embodiment in the construct the polynucleotide sequence is operably linked to a heterologous promoter.

In one embodiment in the construct is a plant expression vector containing the LtP1-L or LeP1-L polynucleotide.

Preferably the LtP1-L polynucleotide encodes amino acid sequence of LtP1-L protein as shown in SEQ ID No. 2. Preferably the LtP1-L polynucleotide comprises nucleotide sequence of LtP1-L as shown in SEQ ID No. 1.

Preferably the LeP1-L polynucleotide encodes amino acid sequence of LeP1-L protein as shown in SEQ ID No. 6. Preferably the LeP1-L polynucleotide comprises nucleotide sequence of LeP1-L as shown in SEQ ID No. 5.

Genetic constructs of the present invention comprise one or more polynucleotide sequences of the invention and/or polynucleotides encoding polypeptides of the invention, and may be used for transforming, for example, bacterial, fungal, insect, mammalian or plant organisms.

Methods for producing and using genetic constructs and vectors are well known in the art and are described generally in Sambrook et al., Molecular Cloning: A Laboratory Manual, 2nd Ed. Cold Spring Harbor Press, 1987; Ausubel et al., Current Protocols in Molecular Biology, Greene Publishing, 1987).

Genetic constructs for expression of genes in transgenic plants typically include promoters for driving the expression of one or more cloned polynucleotide, terminators and selectable marker sequences to detect presence of the genetic construct in the transformed plant.

Suitable promoters which operate in plants include the Cauliflower Mosaic Virus 35S (CaMV 35S). Other examples are disclosed at pg. 120 of Lindsey & Jones (1989) “Plant Biotechnology in Agriculture” Pub. OU Press, Milton Keynes, UK. The promoter may be selected to include one or more sequence motifs or elements conferring developmental and/or tissue-specific regulatory control of expression. Inducible plant promoters include the ethanol induced promoter of Caddick et al (1998) Nature Biotechnology 16: 177-180.

Other examples of constitutive plant promoters include the nopaline synthase promoter and the octopine synthase promoter, and the Ubi 1 promoter from maize.

The promoters suitable for use in the constructs of this invention may be functional in a cell, tissue or organ of a monocot or dicot plant and include cell-, tissue- and organ-specific promoters, cell cycle specific promoters, temporal promoters, inducible promoters, constitutive promoters that are active in most plant tissues, and recombinant promoters. Choice of promoter will depend upon the temporal and spatial expression of the cloned polynucleotide, so desired.

The invention provides a host cell which comprises a genetic construct or vector of the invention.

The invention further provides plant cells which comprise a genetic construct of the invention, and plant cells modified to alter expression of a polynucleotide or polypeptide of the invention, or used in the methods of the invention. Plants comprising such cells also form an aspect of the invention.

In a further embodiment the invention provides a cell comprising a LtP1-L or LeP1-L polynucleotide of the invention, preferably the cell is transgenic for the polynucleotide. Preferably the transgenic cell, is transformed to comprise the LtP1-L or LeP1-L polynucleotide of the invention. Alternatively, a predecessor of the cell has been transformed to comprise the LtP1-L or LeP1-L polynucleotide, and the cell is an off-spring of the predecessor cell and has inherited the polynucleotide that was transformed into the predecessor cell.

In a further embodiment the invention provides a cell comprising a genetic construct of the invention.

In a preferred embodiment the cell expresses the LtP1-L or LeP1-L polynucleotide of the invention.

In a preferred embodiment the cell expresses the LtP1-L or LeP1-L protein of the invention.

In a preferred embodiment the cell is transformed or genetically modified to expresses the LtP1-L or LeP1-L polynucleotide or LtP1-L or LeP1-L protein of the invention.

In one embodiment the cell is selected from a bacterial cell, a yeast cell, a fungal cell, an insect cell, algal cell, and a plant cell. In one embodiment the cell is a bacterial cell.

In one embodiment the cell is a plant cell e.g. a duckweed plant cell.

Following transformation of a plant cell, a plant may be regenerated, e.g. from single cells, callus tissue or leaf discs, as is standard in the art. Almost any plant can be entirely regenerated from cells, tissues and organs of the plant. Available techniques are very well known in the art and are reviewed in Vasil et al., Cell Culture and Somatic Cell Genetics of Plants, Vol I, II and III, Laboratory Procedures and Their Applications, Academic Press, 1984, and Weissbach and Weissbach, Methods for Plant Molecular Biology, Academic Press, 1989.

Preferably the plant is transgenic for the polynucleotide. Preferably the transgenic plant is transformed to comprise the polynucleotide of the invention. Alternatively, a predecessor of the plant has been transformed to comprise the polynucleotide, and the plant is an off-spring of the predecessor plant and has inherited the polynucleotide that was transformed into the predecessor plant.

In a further embodiment the invention provides a plant comprising a genetic construct of the invention.

In a preferred embodiment the plant expresses the polynucleotide of the invention.

In a preferred embodiment the plant expresses the polypeptide of the invention.

In a preferred embodiment the plant is transformed or genetically modified to expresses the polynucleotide or polypeptide of the invention.

In one embodiment the plant comprises a plant cell of the invention.

The plant will have the modified flavonoid biosynthesis e.g. increased content of orientin and isoorientin, as discussed herein.

In one aspect there is provided a transgenic duckweed plant having a high content of orientin and/or isoorientin obtainable by transforming LtP1-L or LeP1-L into the duckweed, overexpressing LtP1-L or LeP1-L, and obtaining a transgenic duckweed (genetically engineered plant) with higher content of orientin and/or isoorientin than the untransformed e.g. wild-type plant.

In one aspect there is provide transgenic duckweed having a content of orientin and isoorientin of about 0.29% to 0.37% and/or about 0.53% to 0.73% respectively.

In one embodiment the transgenic duckweed has about 0.29% or 0.37%, or about 0.53% or 0.73%, orientin and/or isoorientin respectively.

Also provided by the present invention is a part, propagule or progeny of such plants, comprising the polynucleotide or construct.

The term “plant part” or grammatical equivalents thereof is intended to include any part of a plant, a tissue, an organ, a seed, a fruit, propagules and progeny of a plant.

The term ‘propagule’ means any part of a plant that may be used in reproduction or propagation, either sexual or asexual, including seeds and cuttings.

The plants of the invention may be grown and either selfed or crossed with a different plant strain and the resulting progeny, comprising the polynucleotides or constructs of the invention, and/or expressing the P1-L sequences of the invention, also form an part of the present invention.

Preferably the plants, plant parts, propagules and progeny comprise a polynucleotide or construct of the invention, and/or express a LtP1-L sequence of the invention.

Preferably the part, propagule or progeny is transgenic for the polynucleotide. Preferably the transgenic part, propagule or progeny is transformed to comprise the polynucleotide of the invention. Alternatively, a predecessor of the plant (that provided the part, propagule or progeny) has been transformed to comprise the polynucleotide, and the part, propagule or progeny provided by an off-spring of the predecessor plant and has inherited the polynucleotide that was transformed into the predecessor plant.

In a further embodiment the invention provides a part, propagule or progeny comprising a genetic construct of the invention.

In a preferred embodiment the part, propagule or progeny expresses the polynucleotide of the invention.

In a preferred embodiment the part, propagule or progeny expresses the polypeptide of the invention.

In a preferred embodiment the part, propagule or progeny is transformed or genetically modified to expresses the polynucleotide or polypeptide of the invention.

In one embodiment the part, propagule or progeny comprises a plant cell of the invention.

The part, propagule or progeny of a plant of the invention will have the modified flavonoid biosynthesis e.g. increased content of orientin and isoorientin, as discussed herein.

In one aspect there is provided a process of producing a flavonoid, which is optionally orientin and/or isoorientin in a plant, which method comprises culturing or growing said plant, and isolating the flavonoid from the host, or thereby enriching the flavonoid in host biomass or material.

Thus there is provided a method for producing orientin and/or isoorientin, the method comprising (i) introducing LtP1-L or LeP1-L into the duckweed genome, (ii) overexpressing LtP1-L or LeP1-L, thereby obtaining transgenic duckweed with higher content of orientin and/or isoorientin than wild-type plants; (iii) cultivating the transgenic duckweed, and (iv) extracting orientin and/or isoorientin.

As explained in the Examples below, conventional purification methods can be used to obtain high purity orientin and isoorientin.

Rather than using the duckweed tissue itself, the duckweed culture medium may be obtained by cultivating duckweed, and orientin and/or isoorientin are extracted from the culture medium.

In particular, once the duckweed is removed (e.g. filtered) from the duckweed culture medium, there is obtained a culture aqueous solution, from which orientin and/or isoorientin can bee extracted.

Duckweed may be cultured using an aqueous solution containing sucrose. In an aqueous solution containing sucrose, the concentration of sucrose may be about 1% (weight to volume ratio, unit: g/ml), preferably 2%. On other embodiments it is 1%-5% e.g. 5%.

Example the cultivation conditions include a photoperiod of 16 hours/8 hours (light/dark) and temperature, 25° C.

In one aspect of the invention there is provided a culture solution of transgenic duckweed having a content of orientin and isoorientin of about 16.83 mg/L to 22.38 mg/L and 24.89 mg/L to 30.54 mg/L respectively.

In one embodiment the solution has about 16.83 mg or 22.38 mg/L, or about 24.89 mg/L or 30.54 mg/L respectively.

In one embodiment the solution has a higher concentration (orientin ~104.21 mg/L, isoorientin ~191.44 mg/L). This was achieved with LeP1-L overexpression lines as described below.

In one embodiment the solution is a hydroponic solution.

In one aspect of the invention there is provided a flavonoid, which is optionally orientin and/or isoorientin, obtained or obtainable by the processes described herein e.g. from the plants, plant parts or culture media.

Orientin (FIG. 8b) has antibacterial, antiviral, anti-inflammatory, analgesic, neuroprotective, and cardiac protective effects, and is widely used in clinical practice (Yamaguchi, K. K. D., Pereira, L. F. R., Lamarao, C. V., Lima, E. S., da, Veiga, V. F. (2015). Amazon acai: Chemistry and biological activities: A review. Food Chemistry, 179, 137-151.).

Isoorientin (FIG. 8b) is a flavonoid compound of luteolin glycoside, which exists in many medicinal plants. Isoorientin has various pharmacological activities, such as antioxidant activity, inhibition of inflammation development, improvement of insulin resistance, weakening of liver fibrosis development, and induction of liver cancer cell apoptosis.

In addition, isoorientin has been reported to improve the stability of silver nanoparticles, reduce their toxicity, and enhance the inhibitory effect on a-glucosidase and pancreatic lipase, exhibiting the further development of highly stable and lowly cytotoxic AgNPs-Iso on Type II diabetes and obesity (Wang, X., Yuan, L., Deng, H., Zhang, Z. (2021). Structural characterization and stability study of green synthesized starch stabilized silver nanoparticles loaded with isoorientin. Food Chemistry, 338, 127807). Isoorientin exhibited high photoprotective activity against UV-B (290-320 nm) radiations, revealing its possibility as an antioxidant and sunscreening agent in cosmetic formulations (Lefahal, M., Makhloufi, E. H., Boussetla, A., Ayad, R., Rayane, S. A., Akkal, S. (2022). Isoorientin isolated from the Algerian Halophyte Limonium thouinii (Viv.) kuntze as a multifunctional cosmetic ingredient: Antioxidant and photoprotective effects evaluation. Proceedings of the National Academy Sciences, India Section B: Biological Science 92, 889-896).

Due to their antioxidant, anti proliferative, anticancer, antiviral, and anti-inflammatory properties, they are currently widely used in fields such as pharmaceuticals, food and beverage, dietary supplements, and cosmetic additives

At present, the flavonoid compounds orientin and isoorientin in the market are mainly extracted from wild plants, resulting in high production costs. Commercially available orientin and isoorientin are priced at 800 yuan/g. The method of extracting flavonoids by collecting plant resources from nature is no longer sufficient to meet the growing market demand. Therefore, seeking methods that are easy to scale and automate the production of flavonoids is an important issue that urgently needs to be addressed.

In one aspect of the invention there is provided use of any of genetically engineered duckweed or its culture medium or orientin and/or isoorientin obtained from either in any of the aforementioned process, which is optionally an industrial process e.g. in a food or feed product, a health product, or a cosmetic product.

Such food or feed products, health products, or cosmetic products, form further aspects of the invention.

The following definitions and descriptions are made to better explain the aspects and embodiments of the invention:

### Methods for Modifying Endogenous Polynucleotides

As explained above, modification of the sequences of the invention by gene editing is also embraced by the present invention.

As shown in the Examples, in certain embodiments of the invention, a genome editing technology (e.g. TALENs, a Zinc finger nuclease or CRISPR-Cas9 technology) can be used to modify one or more base pairs in a target LtP1-L gene. This approach effectively creates a modified allele in the target plant.

The clustered regularly interspaced short palindromic repeat (CRISPR)/CRISPR associated protein (Cas) system is an adaptive immune mechanism found in bacterial and archaeal species that allows the host to combat pathogens, such as bacteriophages (Barrangou, R. et al. Science 315, 1709-1712 (2007); Marraffini, L. A. & Sontheimer, E. J. Science 322, 1843-1845 (2008); Bhaya, D., Davison, M. & Barrangou, R. Annual review of genetics 45, 273-297 (2011); Gameau, J. E. et al. Nature 468, 67-71 (2010)). Bacteriophage-derived 30-bp DNA fragments are inserted into the CRISPR locus of the host cell and transcribed as CRISPR RNAs (crRNAs). These form a complex with trans-encoded RNA (tracrRNA) and CRISPR-associated (Cas) proteins, and the complex introduces site-specific cleavage at DNA sites that match the sequence of the crRNAs.

CRISPR-Cas9 is a type II CRISPR-Cas system. CRISPR-Cas9 system from Streptococcus pyogenes is used in the art as a simple and versatile tool for RNA guided genome editing (RGE) in different organisms. In Cas9 mediated RGE, a single or duplex short RNA molecule (guide RNA or gRNA) directs Cas9 to target the desired DNA site for genome modification or transcriptional control. gRNA-Cas9 recognizes targeted DNA by gRNA-DNA pairing between 5′-end leading sequence of gRNA (referred as gRNA spacer) and one DNA strand (complementary stand of protospacer). Cas9 also requires the presence of protospacer-adjacent motif (PAM) in the target site following the gRNA-DNA pairing region. The approximate 20 nt long gRNA spacer sequence could be readily programmed to target DNA sites with PAM. But gRNA-Cas9 also recognizes PAM sites that match gRNA spacer imperfectly, resulting off-target risk in genome editing. As a result, designing gRNA with highly specific spacer sequence is critical for RGE.

CRISPR-cas9 plasmids for use in plants are commercially available, for example from Addgene—see: www.addgene.org/crispr/plant/

In the context of the present inventions, the LtP1-L gene may be a target for editing using CRISPR-cas9 plasmids (i.e. be used to provide “gRNAs”)—for example to increase native expression by modification of the LtP1-L gene promoter.

### Constructs, Vectors and Components Thereof

The term “genetic construct” refers to a polynucleotide molecule, usually double-stranded DNA, which may have inserted into it another polynucleotide molecule (the insert polynucleotide molecule) such as, but not limited to, a cDNA molecule. A genetic construct may contain the necessary elements that permit transcribing the insert polynucleotide molecule, and, optionally, translating the transcript into a polypeptide. The insert polynucleotide molecule may be derived from the host cell, or may be derived from a different cell or organism and/or may be a recombinant polynucleotide. Once inside the host cell the genetic construct may become integrated in the host chromosomal DNA.

The genetic construct may be a “vector”.

The term “vector” refers to a polynucleotide molecule, usually double stranded DNA, which is used to transport the genetic construct into a host cell. The vector may be capable of replication in at least one additional host system, such as E. coli.

The term “expression construct” refers to a genetic construct that includes the necessary elements that permit transcribing the insert polynucleotide molecule, and, optionally, translating the transcript into a polypeptide. An expression construct typically comprises in a 5′ to 3′ direction:
a) a promoter functional in the host cell into which the construct will be transformed,
b) the polynucleotide to be expressed, and
c) a terminator functional in the host cell into which the construct will be transformed.

The term “coding region” or “open reading frame” (ORF) refers to the sense strand of a genomic DNA sequence or a cDNA sequence that is capable of producing a transcription product and/or a polypeptide under the control of appropriate regulatory sequences. The coding sequence may, in some cases, identified by the presence of a 5′ translation start codon and a 3′ translation stop codon. When inserted into a genetic construct, a “coding sequence” is capable of being expressed when it is operably linked to promoter and terminator sequences.

“Operably-linked” means that the sequenced to be expressed is placed under the control of regulatory elements that include promoters, tissue-specific regulatory elements, temporal regulatory elements, enhancers, repressors and terminators.

The term “noncoding region” refers to untranslated sequences that are upstream of the translational start site and downstream of the translational stop site. These sequences are also referred to respectively as the 5′ UTR and the 3′ UTR. These regions include elements required for transcription initiation and termination, mRNA stability, and for regulation of translation efficiency.

Terminators are sequences, which terminate transcription, and are found in the 3′ untranslated ends of genes downstream of the translated sequence. Terminators are important determinants of mRNA stability and in some cases have been found to have spatial regulatory functions.

The term “promoter” refers to non-transcribed cis-regulatory elements upstream of the coding region that regulate gene transcription. Promoters comprise cis-initiator elements which specify the transcription initiation site and conserved boxes such as the TATA box, and motifs that are bound by transcription factors. Introns within coding sequences can also regulate transcription and influence post-transcriptional processing (including splicing, capping and polyadenylation).

A promoter may be homologous with respect to the polynucleotide to be expressed. This means that the promoter and polynucleotide are found operably linked in nature.

Alternatively the promoter may be heterologous with respect to the polynucleotide to be expressed. This means that the promoter and the polynucleotide are not found operably linked in nature.

In certain embodiments the P1-L polynucleotides/polypeptides of the invention may be advantageously expressed under the control of selected promoter.

The promoters may be derived from genes of other plants, viruses, and plant pathogenic bacteria and fungi. Those skilled in the art will, without undue experimentation, be able to select promoters that are suitable for use in modifying and modulating plant traits using genetic constructs comprising the polynucleotide sequences of the invention.

The term “polynucleotide(s),” as used herein, means a single or double-stranded deoxyribonucleotide or ribonucleotide polymer of any length but preferably at least 15 nucleotides, and include as non-limiting examples, coding and non-coding sequences of a gene, sense and antisense sequences complements, exons, introns, genomic DNA, cDNA, pre-mRNA, mRNA, rRNA, siRNA, miRNA, tRNA, ribozymes, recombinant polypeptides, isolated and purified naturally occurring DNA or RNA sequences, synthetic RNA and DNA sequences, nucleic acid probes, primers and fragments.

A “fragment” of a polynucleotide sequence provided herein is a subsequence of contiguous nucleotides.

The term “polypeptide”, as used herein, encompasses amino acid chains of any length but preferably at least 5 amino acids, including full-length proteins, in which amino acid residues are linked by covalent peptide bonds. Polypeptides of the present invention, or used in the methods of the invention, may be purified natural products, or may be produced partially or wholly using recombinant or synthetic techniques.

A “fragment” of a polypeptide is a subsequence of the polypeptide that in some embodiments performs a function/activity of and/or influences three dimensional structure of the polypeptide.

The term “isolated” as applied to the polynucleotide or polypeptide sequences disclosed herein is used to refer to sequences that are removed from their natural cellular environment. The isolated sequence is preferably separated from the sequences that may be found flanking the sequence in its naturally occurring environment. An isolated molecule may be obtained by any method or combination of methods including biochemical, recombinant, and synthetic techniques.

The term “recombinant” refers to a polynucleotide sequence that is removed from sequences that surround it in its natural context and/or is recombined with sequences that are not present in its natural context.

A “recombinant” polypeptide sequence is produced by translation from a “recombinant” polynucleotide sequence.

The term “derived from” with respect to polynucleotides or polypeptides of the invention being derived from a particular genera or species, means that the polynucleotide or polypeptide has the same sequence as a polynucleotide or polypeptide found naturally in that genera or species. The polynucleotide or polypeptide, derived from a particular genera or species, may therefore be produced synthetically or recombinantly

As used herein, the term “variant” refers to polynucleotide or polypeptide sequences different from the specifically identified sequences, wherein one or more nucleotides or amino acid residues is deleted, substituted, or added. Variants may be naturally occurring allelic variants, or non-naturally occurring variants. Variants may be from the same or from other species and may encompass homologues, paralogues and orthologues. In certain embodiments, variants of the inventive polypeptides and polypeptides possess biological activities that are the same or similar to those of the inventive polypeptides or polypeptides. The term “variant” with reference to polypeptides and polypeptides encompasses all forms of polypeptides and polypeptides as defined herein.

Variant polynucleotide sequences preferably exhibit at least 70%, more preferably at least 71%, more preferably at least 72%, more preferably at least 73%, more preferably at least 74%, more preferably at least 75%, more preferably at least 76%, more preferably at least 77%, more preferably at least 78%, more preferably at least 79%, more preferably at least 80%, more preferably at least 81%, more preferably at least 82%, more preferably at least 83%, more preferably at least 84%, more preferably at least 85%, more preferably at least 86%, more preferably at least 87%, more preferably at least 88%, more preferably at least 89%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, and most preferably at least 99% identity to a sequence of the present invention (e.g. SEQ ID NO: 1). Identity is found over a comparison window over the entire length of a polynucleotide of the invention.

A preferred method for calculating polynucleotide % sequence identity is based on aligning sequences to be compared using Clustal X (Jeanmougin et al., 1998, Trends Biochem. Sci. 23, 403-5.)

Changes in a native sequence (e.g. SEQ ID NO: 1, SEQ ID NO: 5) may be desirable for a number of reasons. For instance, they may introduce or remove restriction endonuclease sites or alter codon usage. This may be particularly desirable where the genes are to be expressed in alternative hosts e.g. microbial hosts such as yeast. Methods of codon optimizing genes for this purpose are known in the art (see e.g. Elena, Claudia, et al. “Expression of codon optimized genes in microbial systems: current industrial applications and perspectives.” Frontiers in microbiology 5 (2014)). Thus sequences described herein including codon modifications to maximise yeast expression represent specific embodiments of the invention.

Polynucleotide sequence alterations resulting in conservative substitutions of one or several amino acids in the encoded polypeptide sequence without significantly altering its biological activity are also included in the invention. A skilled artisan will be aware of methods for making phenotypically silent amino acid substitutions (see, e.g., Bowie et al., 1990, Science 247, 1306).

The term “variant” with reference to polypeptides encompasses naturally occurring, recombinantly and synthetically produced polypeptides. Variant polypeptide sequences preferably exhibit at least 70%, more preferably at least 71%, more preferably at least 72%, more preferably at least 73%, more preferably at least 74%, more preferably at least 75%, more preferably at least 76%, more preferably at least 77%, more preferably at least 78%, more preferably at least 79%, more preferably at least 80%, more preferably at least 81%, more preferably at least 82%, more preferably at least 83%, more preferably at least 84%, more preferably at least 85%, more preferably at least 86%, more preferably at least 87%, more preferably at least 88%, more preferably at least 89%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, and most preferably at least 99% identity to a sequences of the present invention. Identity is found over a comparison window of at least 20 amino acid positions, preferably at least 50 amino acid positions, more preferably at least 100 amino acid positions, and most preferably over the entire length of a polypeptide of the invention (e.g. SEQ ID NO: 1).

A preferred method for calculating polypeptide % sequence identity is based on aligning sequences to be compared using Clustal X (Jeanmougin et al., 1998, Trends Biochem. Sci. 23, 403-5.)

Changes to a sequence may produce a derivative by way of one or more (e.g. several) of addition, insertion, deletion or substitution of one or more nucleotides in the nucleic acid, leading to the addition, insertion, deletion or substitution of one or more (e.g. several) amino acids in the encoded polypeptide.

Such changes may modify sites which are required for post translation modification such as cleavage sites in the encoded polypeptide; motifs in the encoded polypeptide for phosphorylation etc. Leader or other targeting sequences (e.g. membrane or golgi locating sequences) may be added to the expressed protein to determine its location following expression if it is desired to isolate it from a microbial system.

Other desirable mutations may be random or site-directed mutagenesis in order to alter the activity (e.g. specificity) or stability of the encoded polypeptide. Changes may be by way of conservative variation, i.e. substitution of one hydrophobic residue such as isoleucine, valine, leucine or methionine for another, or the substitution of one polar residue for another, such as arginine for lysine, glutamic for aspartic acid, or glutamine for asparagine. As is well known to those skilled in the art, altering the primary structure of a polypeptide by a conservative substitution may not significantly alter the activity of that peptide because the side-chain of the amino acid which is inserted into the sequence may be able to form similar bonds and contacts as the side chain of the amino acid which has been substituted out. This is so even when the substitution is in a region which is critical in determining the peptides conformation. Also included are variants having non-conservative substitutions. As is well known to those skilled in the art, substitutions to regions of a peptide which are not critical in determining its conformation may not greatly affect its activity because they do not greatly alter the peptide's three dimensional structure. In regions which are critical in determining the peptides conformation or activity such changes may confer advantageous properties on the polypeptide. Indeed, changes such as those described above may confer slightly advantageous properties on the peptide e.g. altered stability or specificity.

A “transgene” is a polynucleotide that is introduced into an organism by transformation. The transgene may be derived from the same species or from a different species to the organism into which the transgene is introduced. In one embodiment the transgene is a naturally occurring sequence. In a further embodiment the transgene is a non-naturally occurring sequence. The transgene may be synthesized or produced by recombinant methods.

“Host cells” may be derived from, for example, bacterial, fungal, yeast, insect, mammalian, algal or plant organisms. Host cells may also be synthetic cells. Preferred host cells are eukaryotic cells. A particularly preferred host cell is a plant cell, particularly a plant cell in a tissue of a plant.

A “transgenic plant” refers to a plant which contains new genetic material as a result of genetic manipulation or transformation. The new genetic material may be derived from a plant of the same species as the resulting transgenic plant or from a different species. Subsequent offspring or generations of the plant that still contain the new genetic material are also transgenic plants according to the invention.

The term “heterologous” (or non-naturally occurring) is used broadly herein to indicate that the gene/sequence of nucleotides in question, or encoded polypeptide (e.g. isoflavone- or other flavonoid biosynthesis modifying polypeptides) have been introduced into said cells of the host or an ancestor thereof, using genetic engineering, i.e. by human intervention. Nucleic acid heterologous to a host cell will be non-naturally occurring in cells of that type, variety or species. Thus the heterologous nucleic acid may comprise a coding sequence of or derived from a particular type of plant cell or species or variety of plant, placed within the context of a plant cell of a different type or species or variety of plant. A further possibility is for a nucleic acid sequence to be placed within a cell in which it or a homologue is found naturally, but wherein the nucleic acid sequence is linked and/or adjacent to nucleic acid which does not occur naturally within the cell, or cells of that type or species or variety of plant, such as operably linked to one or more regulatory sequences, such as a promoter sequence, for control of expression.

A number of patents and publications are cited herein in order to more fully describe and disclose the invention and the state of the art to which the invention pertains. Each of these references is incorporated herein by reference in its entirety into the present disclosure, to the same extent as if each individual reference was specifically and individually indicated to be incorporated by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Thus, for example, reference to “a pharmaceutical carrier” includes mixtures of two or more such carriers, and the like.

Ranges are often expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

Any sub-titles herein are included for convenience only, and are not to be construed as limiting the disclosure in any way.

The invention will now be further described with reference to the following non-limiting Figures and Examples. Other embodiments of the invention will occur to those skilled in the art in the light of these.

The disclosure of all references cited herein, inasmuch as it may be used by those skilled in the art to carry out the invention, is hereby specifically incorporated herein by cross-reference.

The present invention will be further explained in conjunction with embodiments below. However, the scope of the present invention is not limited to the following embodiments. Technicians in this field can understand that various changes and modifications can be made to the present invention without departing from its spirit and scope. Thus the embodiments are provided to fully disclose and describe how to implement and use the claimed implementation scheme, rather than limiting the scope of disclosure in this article. For those skilled in the art, obvious modifications will fall within the scope of the attached claims.

### FIGURES

FIG. 1. Functional characterization of LtP1L. a Phylogenetic tree was constructed using full-length protein sequences of LtP1L and its homologues from eight plant species. Three groups are shown in different colors. b Loss-of-function mutants of LtP1L generated by CRISPR/Cas9. Selected target 1 (T1) and target 2 (T2) are located in the second and third exons of LtP1L, respectively. Sequencing analysis revealed a T insertion at T1 and a T insertion at T2 in the mutants, both of which resulted in a frameshift and premature termination of protein translation. c qRT-PCR analysis of LtP1L transcript abundance in the wild type (WT) and LtP1L overexpression lines. d Phenotype of the WT and transgenic duckweed in the SH medium for two weeks. The bottom lane showed a representative frond for each genotype. e Quantification of total flavonoids in the WT and transgenic duckweed. f Quantification of orientin and isoorientin in the WT and transgenic duckweed. DW, dry weight. Bars represent standard deviation (SD) from three biological replicates. Asterisks denote significant difference between the transgenic duckweed and WT (*, p<0.05; *, p<0.01).

FIG. 2. LtP1L encodes a R2R3-MYB TF with transcriptional activity. a Protein sequence alignments of LtP1L, LeP1L and their homologues. The members of subgroup 7 including AtMYB11, AtMYB12 and AtMYB111 contain SG7 and SG7-2 motifs in the activation domain. The R2 and R3 domains, and SG7 and SG7-2 motifs were underlined. b Subcellular localization of LtP1L. LtP1L-GFP was transiently expressed in Arabidopsis leaf protoplasts. The localization of LtP1L was detected by green fluorescence. The nuclei was marked by NLS-mCherry with red fluorescence. Bar=5 μm. c Transcription activity of LtP1L in yeast. LtP1L fused with the GAL4 DNA binding domain was transformed into yeast AH109, and the transcriptional activity of LtP1L was determined by the detection of two reporter genes HIS and MEL1. d Protein sequence alignment of LeP1L and LtP1L: the protein sequences of LeP1L and LtP1L have a high level of similarity (identity=45.05%).

FIG. 3. LtP1L activates flavonoid synthesis-related gene expression. The transcript levels of flavonoid synthesis-related genes including LtCHS (a), LtCHI (b), LtF2H (c), LtF3′H (e), and LtCGT (d) in the WT and transgenic duckweed. 18S rRNA served as an internal reference gene. The effects of LtP1L on the transcriptional levels of LtCHI (f) and LtF2H (g) were examined with the dual-luciferase reporter assay. Bars represent standard deviation (SD) from three biological replicates. Asterisks denote significant difference between the transgenic duckweed and WT (**, p<0.01).

FIG. 4. LtP1L activates the upstream pathway of flavonoid biosynthesis. The transcript levels of LtPAL (a), LtC4H (b), Lt4CL (c), and LtDHS (e) in the transgenic duckweed and WT. 18S rRNA served as the reference gene. d Activation of the Lt4CL promoter by LtP1L in transient expression assay. Bars represent standard deviations (SD) from three biological replicates. Asterisks represent significant difference between the transgenic duckweed and WT (**, p<0.01).

FIG. 5. Functional characterization of LtABCC4. a Phylogenetic analysis of nineteen ABCC transporters from four different species. b Loss-of-function mutants of the LtABCC4 generated by CRISPR/Cas9. Selected target 1 (T1) and target 2 (T2) are located in the first and second exons of the LtABCC4, respectively. Sequencing analysis revealed a C insertion at T1 and an A insertion at T2 in the mutants, both of which resulted in a frameshift and premature termination of protein translation. c Phenotype of Ltabcc4^{crispr}-1, and Ltabcc4^{crispr}-2 grown in the SH medium for two weeks. d Quantification of total flavonoids in the WT and transgenic duckweed. e Quantification of orientin and isoorientin in the WT and transgenic duckweed. DW, dry weight. Bars represent standard deviation (SD) from three biological replicates. Asterisks represent significant difference between the transgenic duckweed and WT (**, p<0.01).

FIG. 6. LtP1L activates the expression of LtABCC4. a Subcellular localization of LtABCC4 in Arabidopsis protoplast. The green fluorescence of LtABCC4-GFP was merged with the red fluorescence of a tonoplast mark (AtTiP1;1-mCherry). Bar=5 μm. b The transcript levels of LtABCC4 in the transgenic duckweed and WT. 18S rRNA was used as an internal reference gene. c Activation of LtABCC4 by LtP1L in transient expression assay. LtP1L as an effector was used to transfect Arabidopsis protoplasts along with the LtABCC4 promoter fused with luciferase reporter. d Binding of the LtABCC4 promoter by LtP1L via yeast one-hybrid assay. The LtABCC4 promoter (~2.0 kb) was divided into several fragments and recombined into pHIS2.1 vector. Each recombinant construct was transformed into the yeast strain Y187 along with pGADT7-LtP1L. The nails in different colors represent potential cis-elements recognized by LtP1L. e The transcriptional activity of LtABCC4 by LtP1L upon the AC-like cis-element deletion by the luciferase reporter assay. LtABCC4 promoter Δ1 indicated the deletion of AC-like 1, and LtABCC4 promoter Δ2 indicated the deletion of AC-like 2 and 3. f Binding of AC-like cis-element by LtP1L via the EMSA. LtP1L bound to the double stranded DNA probe 1 labeled with biotin, while the binding was competed out by the unlabeled probes 1-7. Bars represent standard deviation (SD) from three biological replicates. (*, p<0.01).

FIG. 7. The color of the duckweed medium. The initial SH medium (a) and the SH medium after culturing duckweed for two weeks (b: wild type, c: LeP1L-OX-1, d: LeP1L-OX-2) were respectively added to the centrifuge tube.

FIG. 8. Detection of flavonoids in the liquid medium of duckweeds. (a) The chromatogram of flavonoids in duckweed liquid medium. The two most prominent peaks were named Peak 1 and Peak 2, respectively, for subsequent LC-MS analysis. (b) ultraviolet (UV) spectrum and charge-to-mass ratio analysis of Peak 1 and Peak 2. Based on the results in (a) and (b), Peak 1 was considered as isoorientin and Peak 2 was considered as orientin.

FIG. 9. The content of orientin and isoorientin in solution containing 2% sucrose.

FIG. 10. Obtaining and processing of solution containing orientin and isoorientin.

FIG. 11. The detection of orientin and isoorientin in samples during purification process. HPLC is used to detect orientin and isoorientin in samples from various stages of the purification process, including before (a) and after (b) purification, purified isoorientin (c) and orientin (d).

FIG. 12. Proposed working model of LtP1L in flavonoid metabolism in Lemna turionifera. LtP1L is an R2R3-MYB transcription activator, and synergistically activates phenylalanine synthesis, phenylalanine metabolism, and CGF synthesis pathways, achieving multi-node guidance for metabolic flow. Meanwhile, LtP1L also directly regulates the tonoplast transporter LtABCC4, which is responsible for the transport of CGFs into vacuoles. Therefore, LtP1L plays a pivotal role in CGF synthesis and transport in L. turionifera.

FIG. 13. Enhancement of flavonoid accumulation by L. turionifera transgenic plants expressing LeP1L. (a) The determination of total flavonoids. The total flavonoid extract reacted with aluminium nitrate, and then the product was measured for absorbance at 510 nm. Total flavonoid content was calculated by the standard curve established by an analytical standard flavonoid rutin. (b) Two major flavone concentration in transgenic duckweed and WT. Total flavonoid extract was analyzed by HPLC, and each individual compound was quantified by corresponding flavone standard. DW, dry weight. Bar represents standard deviations (SD) from three biological repeats. Asterisks represent the significant difference between transgenic duckweeds and WT (*P<0.05).

FIG. 14. Effects of sucrose solution on the synthesis of CGFs in L. turionifera transgenic over-expressing (“OX”) plants expressing LeP1L. LeP1L-OX lines and wild type were treated with 2% sucrose solution for 7 days. Several indicators related to flavonoids were measured, including the total flavonoid content in plant (a), the content of isoorientin (b) and orientin (c) in plant, the proportion of isoorientin (d) and orientin (e) in total flavonoids, the content of isoorientin (f) and orientin (g) in solution, and the total production of isoorientin (h) and orientin (i) by duckweeds. DW, dry weight. ND, no detected. Bar represents standard deviations (SD) from three biological repeats. Asterisks represent the significant difference between two groups (*P<0.05).

### EXAMPLES

The instruments, reagents, materials, etc. involved in the following embodiments, unless otherwise specified, are conventional instruments, reagents, materials, etc. already existing in the prior art and can be obtained through formal commercial channels. The experimental methods, detection methods, etc. involved in the following embodiments, unless otherwise specified, are all conventional experimental methods, detection methods, etc. existing in the existing technology.

### Example 1: Cloning of LtP1-L from Duckweed

(1) Duckweed culture: duckweed is cultured in the climatron under the conditions of light cycle 16 h/8 h (light/dark), 25° C.

(2) Extraction of total RNA from duckweed leaves: About 100 mg of young duckweed leaf tissue material was fully ground into powder, and the total RNA was extracted according to the instructions of the plant total RNA extraction kit (Transgen, Beijing); 3 μL total RNA was subjected to agarose gel electrophoresis to identify the quality of total RNA, and then the concentration of total RNA was measured on NanoDrop (Thermo Fisher, USA) spectrophotometer.

(3) Gene cloning: Using the extracted total RNA as a template (500 ng), the first strand cDNA was produced according to the instructions of the PrimeScript 1st Strand cDNA Synthesis Kit (TaKaRa, Dalian); LtP1-L was amplified using cDNA with specific primers. The specific primer sequences are as follows:

Forward primer P1:
5′-ATGGGGAGAGCCTTGCTG-3′;
Reverse primer P2:
5′-TCAGAGGATCCCCCGAGATCT-3′.

PCR reaction system (total volume 50 μL) For: 5 μL 10×KOD buffer, 5 μL dNTPs, 4 μL MgSO4, 1 μL forward primer, 1 μL reverse primer, 1 μL cDNA template, 1 μL KOD enzyme, ddH2O supplemented to 50 μL.

The PCR amplification conditions were: pre-denaturation at 95° C., 3 minutes, 35 cycles at 95° C., 30 seconds; 54° C., 30 seconds; 68° C., 100 seconds, and finally extend for 5 minutes at 68° C.

Recycling and purifying the PCR products.

Through the above method, the LtP1-L was obtained, and its nucleotide sequence is shown in SEQ ID NO. 2. The deduced amino acid sequence of the encoded protein is shown in SEQ ID NO. 1.

### Example 2—Construction of Plant Expression Vector Containing LtP1-L

(1) The plant expression vector pCAMBiaZmUBI1p was linearized through enzyme digestion and then purified.

(2) At 50° C., the LtP1-L was recombined into the plant expression vector pCAMBiaZmUBI1p after 30 min reaction with homologous recombination enzyme (Novozan, Nanjing).

(3) Transforming Escherichia coli DH5 a Positive clone PCR validation was performed on receptive cells to obtain a pCAMBiaZmUBI1p LtP1-L plant expression vector containing LtP1-L.

### Example 3 Obtaining Genetically Engineered Plants

(1) The constructed pCAMBiaZmUBI1p LtP1-L plant expression vector was transfected into Agrobacterium tumefaciens by electric shock, and a positive Agrobacterium tumefaciens strain was obtained through PCR verification.

(2) Shake the overnight cultivated Agrobacterium to OD600≈0.4 and add 100 μM of Acetosyringone, continue to culture for 2 h, centrifugate and collect the cell, resuspend the cell with transformation solution to OD600≈0.4, transfer the duckweed callus into the bacterial solution, vacuumize for 10 min, ultrasonic for 5 min, and then vacuumize for 10 min. Take out the callus tissue, lay it on filter paper, culture it in dark for 3 days, and then transfer it to differentiation medium. After screening, genetically engineered duckweed strains were obtained.

The specific operation is as follows: lay the duckweed leaves flat on the induction medium, callus appeared after 3-4 weeks of dark cultivation at 24° C. Then transfer the callus tissue to a new induction medium for further cultivation for 4-5 weeks for transformation.

The single colony of Agrobacterium tumefaciens with the plant overexpression vector of duckweed LtP1-L gene was transferred to the liquid Lysogeny broth containing 50 mg/L kanamycin and 25 mg/L Rifampicin. Shake the culture at 28° C. (200 rpm) for growth until the OD600 reaches 0.4. Add the newly prepared 100 μM Acetosyringone was added to the culture, and the mixture was shaken for 2 hours. Then 2400 g, centrifuge for 15 minutes to collect the bacterial body, and resuspend in the induction medium. Adjust the density (OD600) of the resuspended Agrobacterium to approximately 0.4 for infecting the callus of duckweed.

Immerse the callus tissue into an Agrobacterium suspension in a 350 mL tissue culture bottle. Place the bottle in a vacuum chamber and apply vacuum for 10 minutes. Then, place the bottle in a bath ultrasonic instrument (Branson Ultrasonic Cleaner CPX2800, Branson Ultrasonics Corp., Danbury, CT, USA), and perform ultrasonic treatment at a frequency of 40 kHz at 17° C. for 5 minutes. After ultrasonic treatment, vacuum permeate the bottle again for 10 minutes. After releasing the vacuum, gently shake the callus tissue block and Agrobacterium to incubate for 30 minutes. After culture, the infected calli were transferred to the filter paper wetted with induction medium and placed in an empty Petri dish in the dark at 25° C. for co culture. After co culturing for three days, transfer the infected callus tissue to regeneration medium. Transfer the regenerated duckweed leaves to a preservation medium containing 0.6% sucrose, waiting for the growth and proliferation of duckweed.

### Example 4: Determination of Orientin and Isoorientin Content in Genetically Engineered Plants

Collect genetically engineered plants cultured for 2 weeks in Example 3 (a total of 2 strains were obtained, named LtP1-LOX-1 and LtP1-LOX-2, respectively), freeze dry and extract total flavonoids using 80% methanol ultrasound. Then, analyze the content of orientin and isoorientin by HPLC. The wild type of duckweed obtained from cultivating non transfected callus tissue was used as a control.

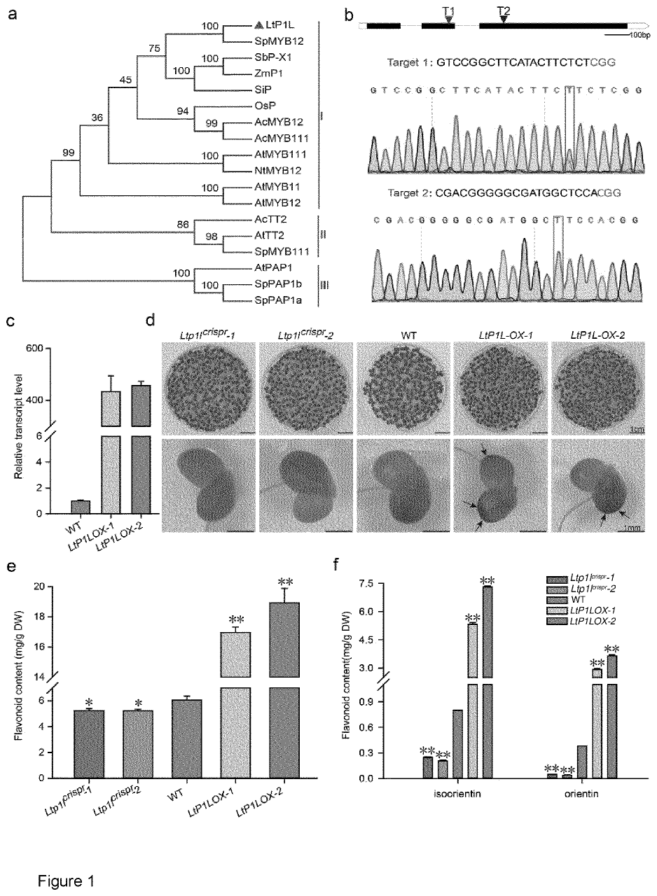

Result: As shown in FIGS. 1e and 1f, for total flavonoids, the content of wild-type is 6.06% (weight of total flavonoids as a percentage of plant dry weight), the content of LtP1-LOX-1 is 16.79%, and the content of LtP1-LOX-2 is 18.93%. For orientin, the content in the wild type is 0.04% (the weight of orientin as a percentage of plant dry weight), the content in LtP1-LOX-1 is 0.29%, and the content in LtP1-LOX-2 is 0.37%. For Isoorientin, the content in the wild type is 0.08% (percentage of Isoorientin weight to plant dry weight), the content in LtP1-LOX-1 is 0.53%, and the content in LtP1-LOX-2 is 0.73%. It can be seen that the content of total flavonoids, orientin, and isoorientin in genetic engineering strains increased by 2.5-3 times compared to the wild type, while the content of orientin and isoorientin increased by 6-10 times, showing a significant increase.

### Example 5: Determination of Orientin and Isoorientin Content in Plant Culture Medium of Genetic Engineering

Cultivate 4 grams of fresh weight wild type duckweed, LtP1-LOX-1, and LtP1-LOX-2 strains (with 3 groups each) in 20 mL of an aqueous solution containing 2% (weight to volume ratio, unit: g/ml) sucrose, under the conditions of a light cycle of 16 hours/8 hours (light/dark) and 25° C. After 7 days of cultivation, filter and remove the duckweed to obtain a culture aqueous solution. Analyze the content of orientin and isoorientin in the culture aqueous solution by HPLC.

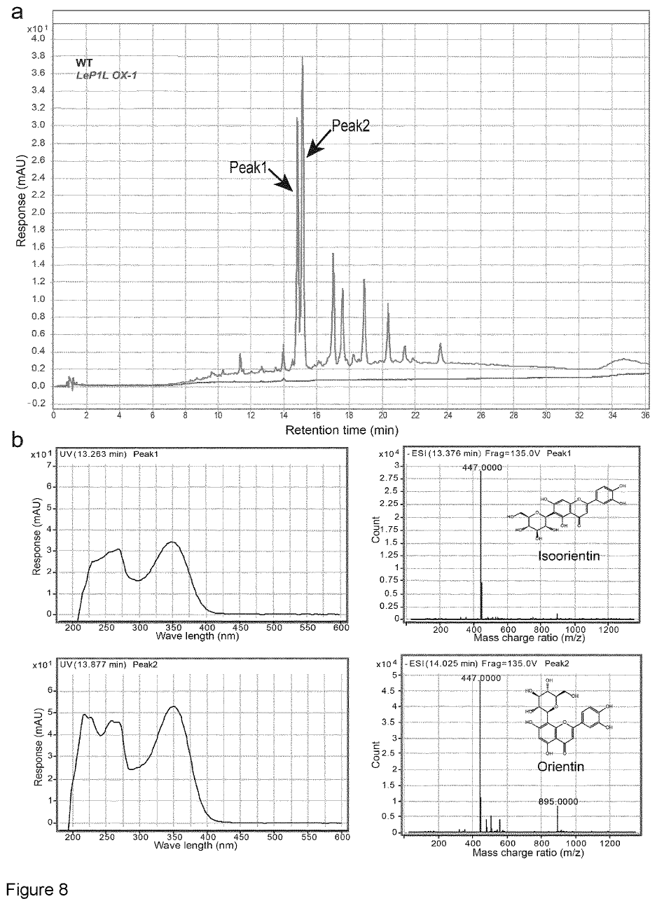

Result: As shown in FIG. 9, in the culture solution of wild type duckweed, the content of orientin is 0.84 mg/L, and the content of isoorientin is 0.73 mg/L; The contents of orientin and isoorientin in LtP1-LOX-1 are 16.83 mg/L and 24.89 mg/L respectively; The contents of orientin and isoorientin in LtP1-LOX-2 are 22.38 mg/L and 30.54 mg/L respectively. It can be seen that the contents of orientin and isoorientin in the culture aqueous solution of each strain are high, and orientin and isoorientin can be directly extracted from the culture aqueous solution. This discovery is a serendipitous discovery. Compared with the extraction from the leaves of duckweed, the extraction steps are greatly simplified, and the extraction cost is significantly reduced.

### Example 6—Further Analysis and Investigation

### Additional Methods

### Gene Cloning, Phylogenetic Analysis, and Sequence Alignment

Unless otherwise stated total RNA was extracted using the PLANTpure Universal RNA Kit (Aidlab, Beijing, China), and genomic DNA was removed with the DNase Digestion Kit (Aidlab).

Gene-specific primers for 5′- or 3′-RACE PCR were designed based on the partial coding sequence of LtABCC4 obtained in our transcriptome data. The full-length cDNA sequence of LtABCC4 was obtained following the supplier's instructions with the SMARTer® RACE 5′/3′ Kit (TaKaRa).

Full-length protein sequences were aligned by ClustalW, and the phylogenetic tree was constructed using the Neighbor-Joining (NJ) algorithms in MEGA 7.0. Evaluation of the nodes' significance was performed by bootstrap analysis with 1,000 replicates. Seven MYB12 homologous protein sequences were aligned by the Bioedit software (V7.0) to identify the conserved domain.

### Vector Construction and Duckweed Transformation

The LtP1L and LtABCC4 coding sequences were recombined into the modified pCAMBIA1300 vector to generate the overexpression construct under control of a maize polyubiquitin gene promoter. A CRIPSR/Cas9 construct carrying two sgRNA cassettes was generated using the binary pYLCRIPSR/Cas9 multiplex genome targeting vector system. Two sgRNA cassettes driven by OsU6a and OsU6b, respectively, were assembled according to the golden gate cloning protocol. The resulting constructs were transformed into Agrobacterium tumefaciens strain EHA105 by electroporation. The A. tumefaciens strain was used to inoculate the embryogenic calli of Lemna turionifera, following our previously reported method, and dozens of transgenic duckweed strains were successfully obtained.

### More Detailed Description of Cultivation and Growth Performance of Transgenic Duckweed

Transgenic lines of L. turionifera and the wild type (WT) were separately cultured with the Schenk & Hildebrandt (SH) liquid medium containing 10 g/L sucrose for two weeks, and then morphological observation of these plants was carried out using an SZX16 stereo microscope (Olympus).

### More Detailed Description of Flavonoid Content and Composition Analysis

Total flavonoids were extracted from freeze-dried duckweed powder using 80% (v/v) methanol. The extraction process included two repeats of 30-min ultrasound sonication with a 10-min interval. After storage at 4° C. overnight, the supernatant was obtained by centrifugation at 12,000 rpm for 5 min, and was regarded as the total flavonoid extract. Total flavonoid content was determined by the method reported before. Briefly, 0.3 mL of 5% sodium nitrite solution was added into 0.5 mL of total flavonoid extract, and then 0.3 mL of 10% (x/x) aluminum nitrate solution was added. After standing for 5 min, the mixture was added with 4 mL of 1 mol/L NaOH, and then made up to 6 mL with 65% (v/v) ethanol solution. The absorbance at 510 nm was measured by spectrophotometry (GeneQuant 1300, GE Healthcare), and the content of total flavonoid was calculated by the standard curve established by a series of concentrations of rutin (Sigma-Aldrich).

For the flavonoid composition analysis, the total flavonoid extract was analyzed by the high-performance liquid chromatography (HPLC) (1260 Infinity II, Agilent) with a Boltimate LP-C18 column (Agilent). Each individual compound was detected by an ultraviolet (UV) detector, and the quantification of these compounds was calculated by comparison of the peak area between the total flavonoid extract and each flavonoid standard orientin and isoorientin (Sigma-Aldrich).

### Subcellular Localization of LtP1L and LtABCC4

The gene coding region was fused with GFP at its C-terminus and expressed under control of a 35S cauliflower mosaic virus (CaMV 35S) promoter in the modified pBI221-GFP vector. Arabidopsis transient expression assay was performed following a method described previously. Protoplasts from four-week-old rosette leaves were transformed via the PEG-mediated method. The fluorescence was observed using a FluoView FV1000 confocal microscope (Olympus, Tokyo, Japan) with an excitation of 488 nm and an emission of 510 nm for GFP, and with an excitation of 552 nm and an emission of 610 nm for mCherry.

### Transcriptional Activity of LtP1L

The LtP1L coding sequence was ligated into the pGBKT7 vector (TaKaRa). The pGBKT7-LtP1L vector and empty vector were transformed into a yeast strain AH109 using Yeastmaker™ Yeast Transformation System 2 (TaKaRa). The proper AH109 strains were inoculated into Minimal Media Single Dropouts (SD)/-Trp, SD/-His, and SD/-Trp plus X-α-gal, and cultured at 30° C. for 3 d.

Quantitative Real Time PCR (qRT-PCR)

The method of RNA extraction and gDNA digestion was the same as described above. The first-strand cDNA was synthesized using TransScript® One-Step gDNA Removal and cDNA Synthesis SuperMix (TransGen, Beijing, China). Primers were designed by Beacon Designer v7.0 (Premier Biosoft). qRT-PCR was conducted on a LightCycler® 480 detection system (Roche) with SYBR Premix Ex Taq (TaKaRa). 18S rRNA was used as an internal reference gene.

### Dual Luciferase Analysis

The promoter sequences of LtABCC4, LtF2H, Lt4CL and LtCHI were cloned by the method reported previously. LtABCC4, LtF2H, Lt4CL, and LtCHI promoter were respectively ligated into the pGreenII 0800-LUC vector, which contains a Photinus pyralis luciferase as the reporter and a Renilla reniformis luciferase driven by a CaMV 35S promoter as the internal reference. The LtP1L coding region was ligated into the modified pBI221-GFP vector to replace the GFP coding region as the effector. Five micrograms of each plasmid for effectors and reporters were used to transform Arabidopsis protoplasts. The transient expression assay was performed as described above. The luciferase activity was measured by a GloMax 20/20 luminometer (Promega) using a Dual Luciferase Reporter Assay Kit (Vazyme, Nanjing, China). For data analysis, the activities of Photinus pyralis luciferase were quantified and normalized to Renilla reniformis luciferase activities.

### Yeast One-Hybrid

The LtABCC1 promoter (~2 kb) was divided into four fragments including PF1, PF2, PF3 and PF4, and each fragment was about 500 bp. PF2 was further divided into five fragments of approximately 125 bp, named as PF2-1, PF2-2, PF2-3, PF2-4, and PF2-5. Each DNA fragment of LtABCC1 promoter was recombined into the pHIS2.1 vector (TaKaRa). The coding region of LtP1L was ligated into pGADT7 vector (TaKaRa), which was transformed into a yeast strain Y187 using Yeastmaker™ Yeast Transformation System 2 (TaKaRa) along with each recombinant pHIS2.1 construct. The proper Y187 strains were then streaked on SD/-Leu/-Trp and SD/-Leu/-Trp/-His medium, respectively.

### Electrophoretic Mobility Shift Assay

The LtP1L coding region was fused in frame with maltose binding protein (MBP) in the pETMALc-H vector. The recombinant protein was expressed in Escherichia coli and purified by amylose resin (NEB, USA). Biotin-labeled synthetic oligonucleotide was annealed with its unlabeled reverse complementary oligonucleotides, and then used as biotin-labeled probe. A Chemiluminescent EMSA Kit (Beyotime, Shanghai, China) was used to perform the DNA-protein binding reaction. Briefly, the labeled probe (0.1 μmol) was incubated with the recombinant protein (500 ng) in binding buffer for 20 min at room temperature. For competition reactions, 1 pmol of unlabeled probe was added. The protein-probe complex and free probe were separated by polyacrylamide gel electrophoresis, and the biotin-labeled probes were detected by BeyoECL Moon solution in the kit.

### Statistical Analysis

The data were presented as the mean±standard deviation in triplicate. SPSS 19.0 was used for statistical analyses, and P values were calculated using the one-way analysis of variance (ANOVA). Asterisks denote significant differences between two groups of data (*P<0.05, **P<0.01).

### Additional Results

### LtP1L Promotes CGFs Accumulation In Vivo

The phylogenetic analysis of the duckweed MYB transcription factor with its orthologues from seven plant species showed that they were divided into three groups (I to III) (FIG. 1a). The duckweed MYB transcription factor was closely classified with AtMYB12 and ZmP1 in group I, and was designated LtP1L Two biallelic mutant lines (Ltp1I^{crispr}-1 and Ltp1I^{crispr}-2) (FIG. 1b) and two LtP1L overexpressing lines (FIG. 1c) were selected for further analyses. Compared with the wild type (WT), the frond of LtP1LOX-1 and LtP1LOX-2 was smaller and discernable with tan pigment deposits on edges after two-week cultivation in the Schenk & Hildebrandt (SH) liquid medium (FIG. 1d). By contrast, the loss of function of LtP1L did not cause significant changes in frond color and morphology (FIG. 1d).

As noted in Example 4, the flavonoid content of L. turionifera was approximately 6% on the basis of dry weight (DW). In comparison, the flavonoid content was dramatically increased by 180% and 212% in the LtP1L overexpression lines, reaching about 17% and 19% in LtP1LOX-1 and LtP1LOX-2, respectively (FIG. 1e).

Accordingly, significant decreases of about 14% in flavonoid content were observed in the LtP1L mutant lines (FIG. 1e). The contents of single glycosyl CGFs (i.e., orientin and isoorientin) were also dramatically enhanced in the LtP1L overexpression lines while remarkably suppressed in the Ltp1I^{crispr}-1 and Ltp1I^{crispr}-2 lines (FIG. 1f). These results suggest that LtP1L plays a critical role in flavonoid, especially CGFs, biosynthesis in duckweed.

### LtP1L is an R2R3-MYB Transcription Activator

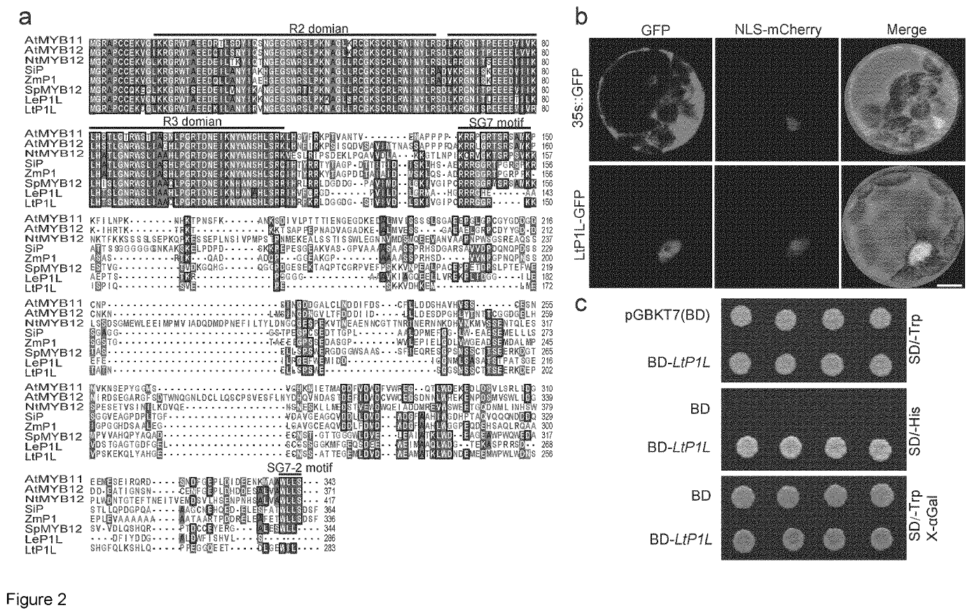

This suggests that LtP1L might have undergone functional divergence. As shown in FIG. 2b, the fluorescence signal of LtP1L-GFP fusion protein was specifically localized in the nucleus, which almost completely overlapped with the signal of the nuclear localization marker mCherry. Moreover, LtP1L enabled the yeast strain AH109 to grow normally on SD/-His medium, and promoted the hydrolysis of X-α-Gal into blue products by activating the expression of MEL1 (FIG. 2c). Therefore, it can be concluded that LtP1L encodes an R2R3-MYB transcription activator involved in the regulation of flavonoid synthesis in duckweed.

### LtP1L Specifically Activates CGF Biosynthesis

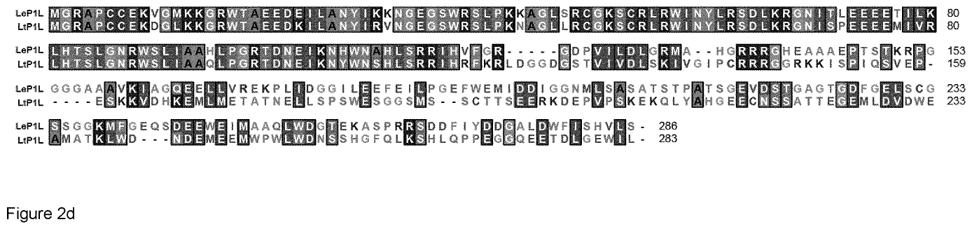

The expression of LtCHS and LtCHI was significantly up-regulated in the LtP1L overexpression lines, while was remarkably down-regulated in the Ltp1I^{crispr}-1 and Ltp1I^{crispr}-2 lines (FIGS. 3a and b). Moreover, the transcription levels of LtF2H, LtCGT, and flavonoid 3′-hydroxylase (F3′H) (FIG. 3c-e), which are responsible for the biosynthesis of CGFs (i.e., orientin and isoorientin), were significantly up-regulated in the LtP1L overexpression lines while was notably down-regulated in the Ltp1I^{crispr}-1 and Ltp1I^{crispr}-2 lines. Additionally, LtP1L drove the transcriptional activity of LtCHI and LtF2H promoters (FIGS. 3f and g). These results correspond to the contents of orientin and isoorientin in the transgenic duckweed lines (FIG. 1f). The magnitude of change in transcription level of LtFLS2, which is involved in flavonol biosynthesis, was significantly reduced compared to other CGFs synthesis related genes in either LtP1L overexpression or Ltp1Icrispr lines (not shown). These results suggest that LtP1L mainly targets genes involved in the metabolism of CGFs.

### LtP1L Regulates Carbon Metabolic Flux Upstream of Flavonoid Metabolism

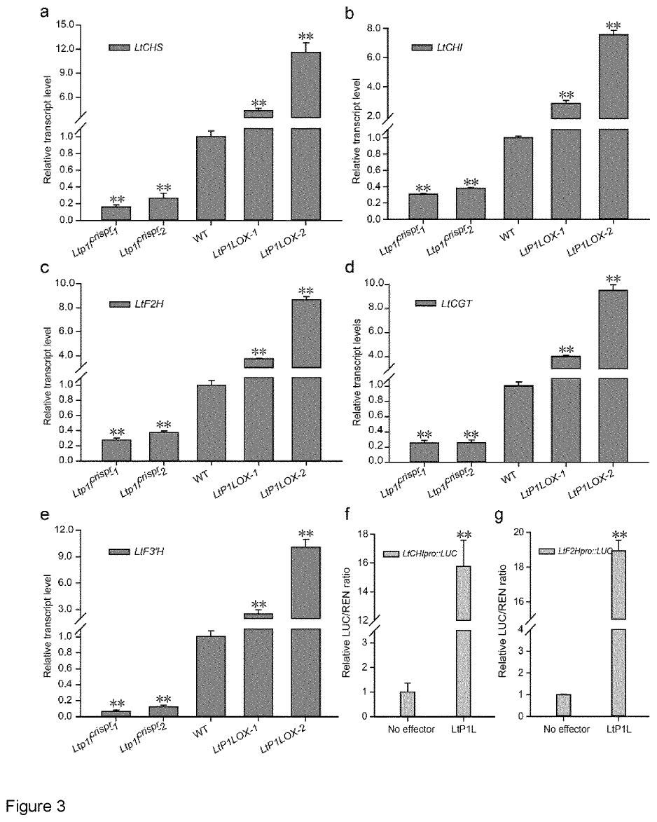

The phenylpropanoid pathway is located upstream of flavonoid metabolism and provides precursors for flavonoid biosynthesis. Our results indicate that LtP1L up-regulates the expression of biosynthesis genes in phenylalanine, phenylpropanoid, and flavonoid pathways. Specifically, the results showed that the expression of phenylalanine ammonium lyase (PAL), cinnamate-4-hydroxylase (C4H), and 4-coumarate:CoA ligase (4CL) was unanimously up-regulated in the LtP1L overexpression lines while was notably down-regulated in the Ltp1I^{crispr}-1 and Ltp1I^{crispr}-2 lines, suggesting that LtP1L promotes the metabolic flux from phenylalanine to the p-coumaroyl CoA (FIG. 4a-d). Additionally, the expression of 3-deoxy-7-phosphoheptulonate synthase (DHS) catalyzing the first committed step in aromatic amino acid biosynthesis, was also up-regulated in the LtP1L overexpression lines while was notably down-regulated in the Ltp1I^{crispr}-1 and Ltp1I^{crispr}-2 lines (FIG. 4e), implying that the LtP1L diverts the carbon flux at the early steps of amino acid metabolism.

### LtABCC4 Encodes a Tonoplast Transporter Involving in CGFs Transportation

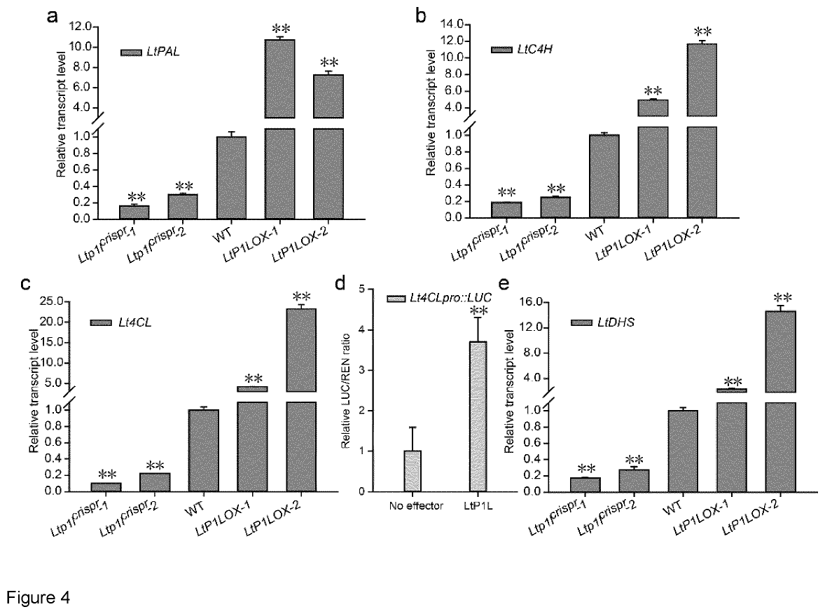

This ABCC protein was named LtABCC4 because it was homologous to CsABCC4a in Saffron and AtABCC4/14 in Arabidopsis as revealed by the phylogenetic analysis (FIG. 5a). Two representative lines Ltabcc^{crispr}-1 and Ltabcc^{crispr}-2 elicited single-base C or A insertion, resulting in frameshift or premature termination of translation, respectively (FIG. 5b). Compared with the WT, loss of function of LtABCC4 caused no discernable changes in frond shape and color (FIG. 5c). However, the loss of function of LtABCC4 resulted in a significant decrease in total flavonoid content, indicating that LtABCC4 is probably involved in the transportation of flavonoids in duckweed (FIG. 5d). Moreover, the content of isoorientin was drastically decreased by 89.86% and 87.27% in Ltabcc4^{crispr}-1 and Ltabcc4^{crispr}-2, respectively (FIG. 5e). However, there was only a slight reduction in orientin content in Ltabcc4^{crispr}-2, while no change was observed in Ltabcc4^{crispr}-1 relative to the WT (FIG. 5e). These results implied that LtABCC4 is involved in CGF transport with substrate favorability for isoorientin.

LtP1L Simultaneously Activates Transport of CGFs into the Vacuole

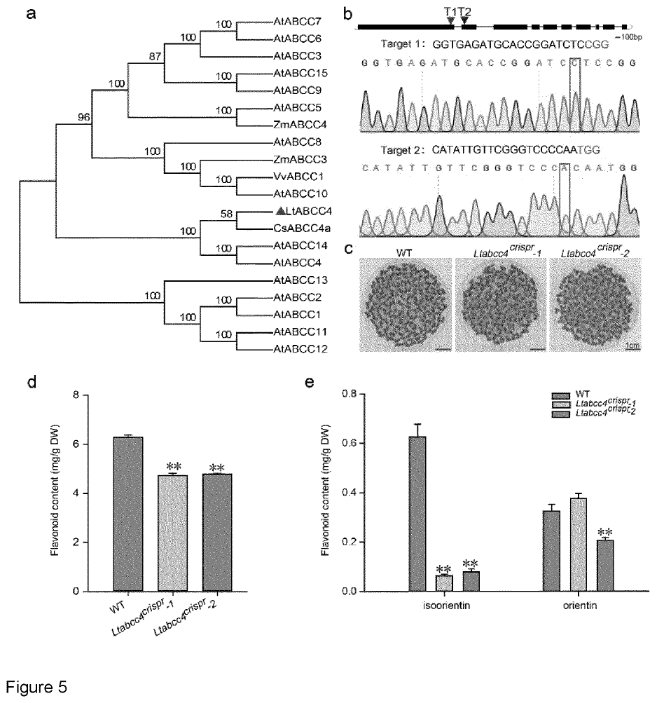

The fluorescence signal of LtABCC4-GFP was almost coincided with the signal of a tonoplast-localized protein γTiP (FIG. 6a), suggesting that LtABCC4 is predominantly localized in the tonoplast. The results of RT-qPCR showed that the transcription level of LtABCC4 was significantly higher in the LtP1L-OX lines and lower in the Ltp1I^{crispr} lines compared with the WT (FIG. 6b). Furthermore, LtP1L as an effector significantly increased the firefly luciferase (LUC) activity, indicating that it drives the transcriptional activity of LtABCC4 promoter (FIG. 6c). The Yeast-One-Hybrid (Y1H) assay showed that LtP1L bound to PF2-5 and activated the HIS3 reporter gene, suggesting that LtP1L binds to AC-like elements in the LtABCC4 promoter (FIG. 6d). In addition, the binding affinity of LtP1L to the LtABCC4 promoter was significantly reduced to 15% or 18%, respectively, upon the deletion of AC-like1 (Δ1) or AC-like2 and 3 (Δ2) cis-elements (FIG. 6e). Furthermore, the electrophoretic mobility shift assay (EMSA) revealed that, LtP1L bound to biotin-labeled DNA probe 1 containing the core sequence of AC-like element was ‘CTACCTAA’ (FIG. 6f). These results suggested that LtP1L directly binds to the promoter of LtABCC4 via a novel AC-like cis-element and activates its transcription. This indicates that LtP1L synergistically regulates the biosynthesis and transportation of CGFs in duckweed.

### LeP1L Enables Duckweeds to Produce Flavonoids, Especially C-Glycosylated Flavones.

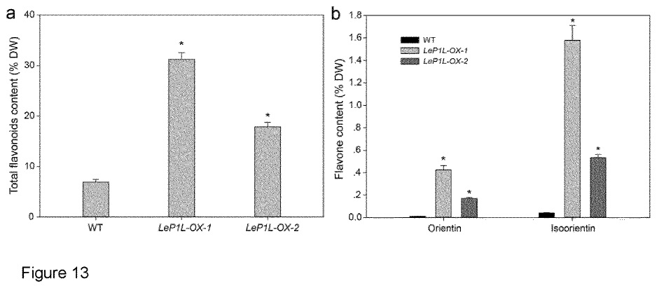

The total flavonoid content of Lemna turionifera was 6.88% dry weight (DV) (FIG. 13a), which is higher than that reported for Landoltia punctata (4.51%) (Huang et al., 2014). The heterologous expression of LeP1L greatly increased the flavonoid content, which reached 31.20% DW in LeP1L-OX-1 and 17.82% DW in LeP1L-OX-2, respectively (FIG. 13a). Given that CGFs are the main component of flavonoid in duckweed, the biosynthesis of two single glycosyl CGFs were also significantly enhanced in transgenic duckweeds (FIG. 13b). The orientin content increased more than 38 times, and isoorientin reached 1.57% DW in LeP1L-OX-1 (FIG. 13b).

### Effects of Sucrose Solution on the Synthesis of CGFs in Duckweeds.

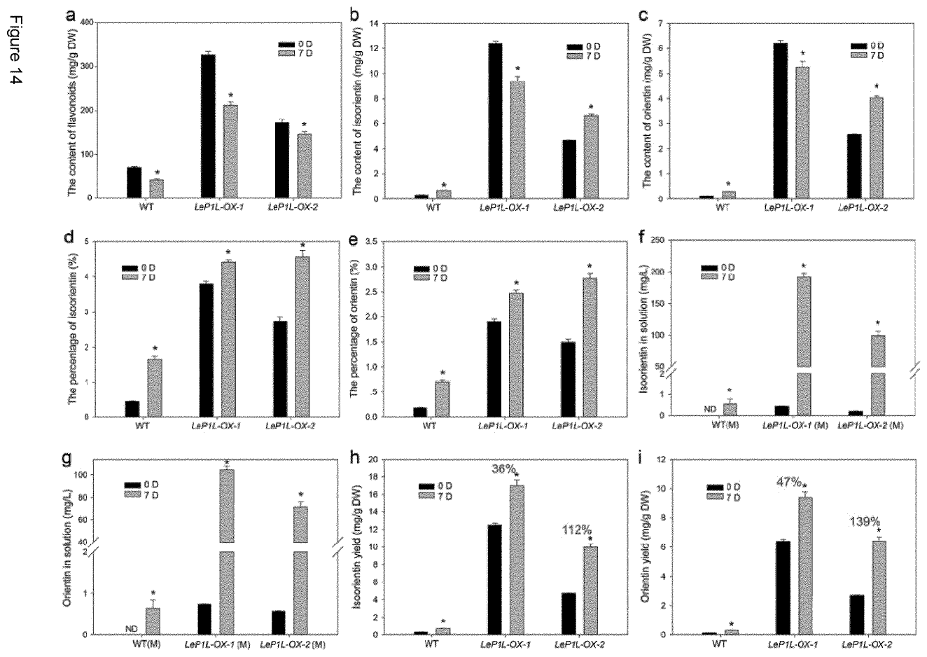

To assess CGF production in LeP1L OX lines, about 4 grams of fresh plant were placed in 20 mL of deionized water containing 2% sucrose under normal culture conditions. After seven-day treatment, transgenic plants and solution were collected separately for evaluating the production efficiency of CGFs. Overall, the total flavonoid content in all plant samples decreased after 7d treatment (FIG. 14a). For the two CGFs, orientin and isoorientin, there was a decrease in the LeP1L OX-1 line (FIGS. 14b and c) which we believe is due to suppression of CGF accumulation in the plant tissues due to the high level of CGFs in the culture solution. There was a significant increase in both the wild-type and LeP1L OX-2 lines (FIGS. 14b and c), which we believe implies the CGF amount in culture solution is not enough to suppress CGF accumulation in plants. However, the proportion of the two CGFs in the total flavonoids were significantly increased in all duckweed samples (FIGS. 14d and e), indicating that the conversion efficiency of the product was significantly improved under 2% sucrose treatment. On the other hand, the contents of orientin and isoorientin in solution were drastically increased in both WT and transgenic duckweeds (FIGS. 14f and g). It was worth noting that the contents of orientin and isoorientin in the LeP1L OX-1 solution were the highest, reaching 104.21 mg/L and 191.44 mg/L, respectively (FIGS. 14f and g). Finally, comprehensive evaluation of the yields of orientin and isoorientin in transgenic duckweed showed that the LeP1L OX-2 line had the largest yield increase, reaching 139% and 112%, and the LeP1L OX-1 line had the highest yield, reaching 9.4 mg/g DW and 17.0 mg/g DW, respectively (FIGS. 14h and i). In conclusion, the synthesis efficiency and productivity of CGFs in duckweeds can be significantly improved by the transgenic methods described herein.

### Overexpression of LeP1L or LtP1L Makes Duckweeds a Novel CGF Production Platform.

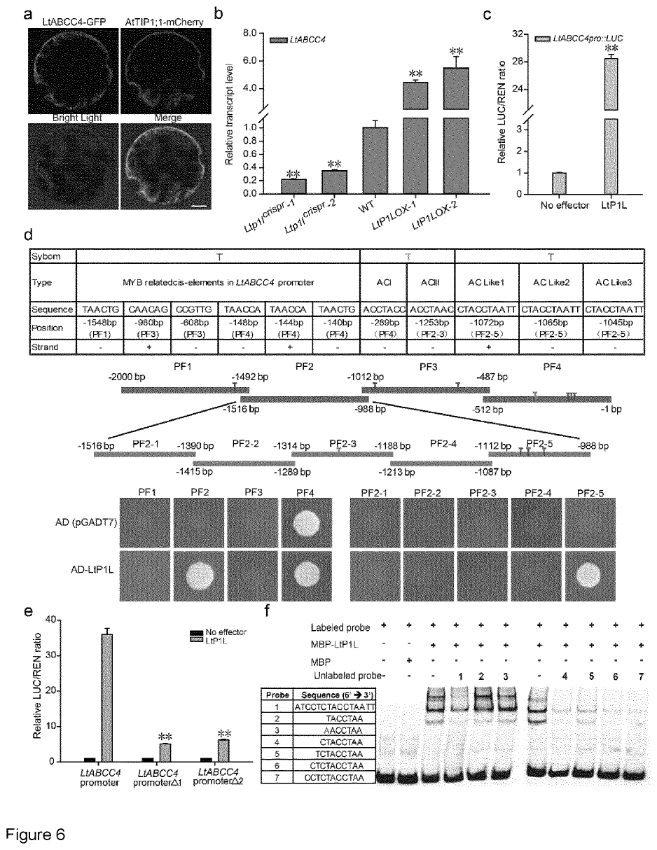

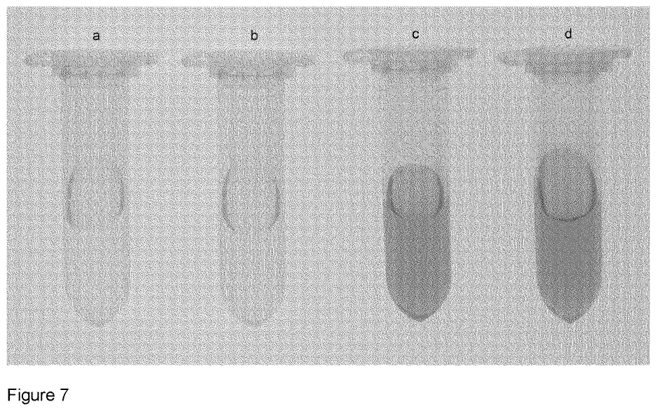

Interestingly, the color of the medium gradually changed during cultivation. After the wild type grew for four weeks, the color of its medium changed from colorless to light yellow (FIGS. 7a and b), while the medium for LeP1L OX-1 and -2 turned brown (FIGS. 7c and d). This phenomenon indicates that the transgenic plants released more colored substances into the apoplast, and then diffused into the medium. Therefore, the composition of flavonoids in the culture medium was analyzed by HPLC, and more than 10 kinds of flavonoids were detected in the medium of transgenic duckweed (FIG. 8a). Subsequently, the UV spectra and mass-to-charge ratios of the two flavonoids with the highest content were further analyzed by LC-MS (FIG. 8b). Taking into account the retention time in HPLC, these two flavonoids were determined to be orientin and isoorientin.

To test the ability of CGFs production in transgenic duckweeds, about 4 grams of fresh plant were placed in 20 mL of deionized water containing 2% sucrose under normal culture conditions. After seven-day treatment, the solution was collected for evaluating the production efficiency of CGFs. AS explained in Example 5, the concentration of orientin was 16.83 mg/L in LtP1L OX-1 and 24.89 mg/L in LtP1L OX-2, while the concentration of isoorientin was 23.38 mg/L in LtP1L OX-1 and 30.54 mg/L in LtP1L OX-2 (FIG. 9). It suggests that LtP1L promoter the release of orientin and isoorientin into solution containing 2% sucrose.

### Culture, Treatment, and Purification of Duckweed

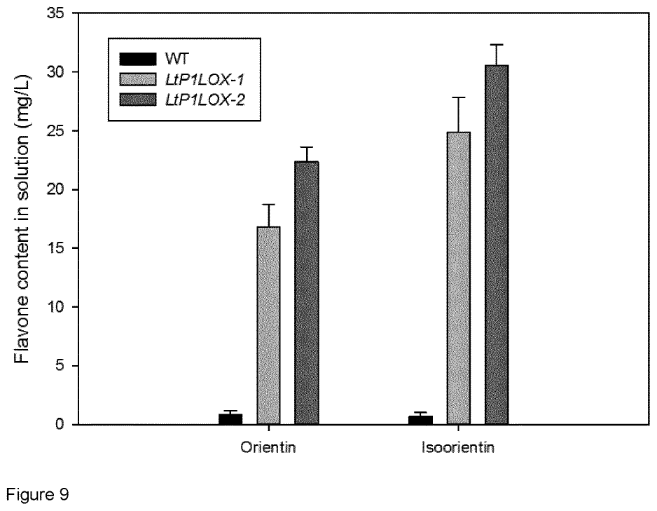

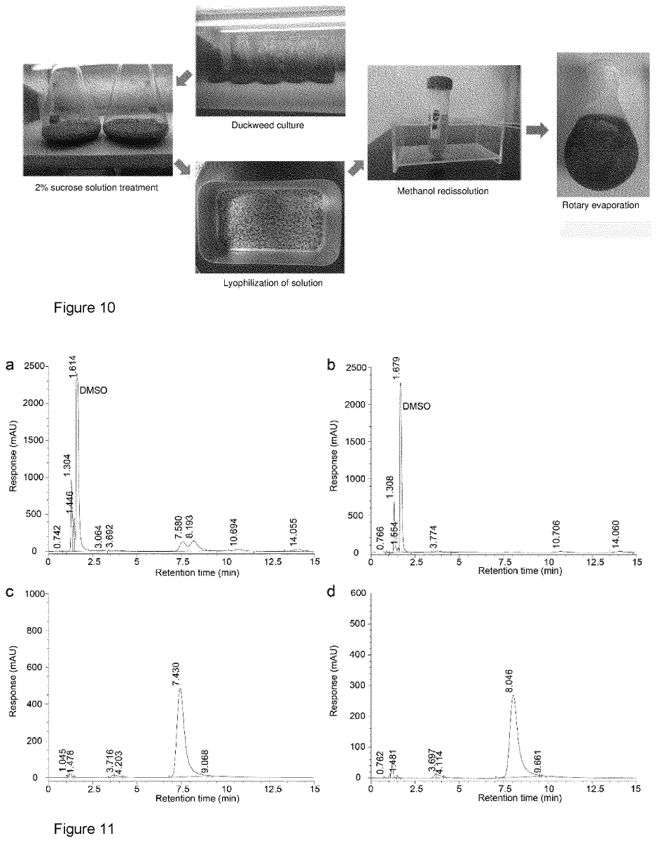

LeP1L OX-1 grown in SH medium for 2 weeks was collected. About 50 g fresh weight duckweed was incubated in 250 mL of 2% sucrose solution for 7 days. A total of 1 L of treatment solution was harvested, freeze-dried, redissolved in 200 mL methanol. The concentration of orientin and isoorientin in the solution was determined by HPLC to be 470.60 mg/L and 648.55 mg/L. Converted to the total amount, it is 94.12 mg of orientin and 129.71 mg of isoorientin, respectively. After evaporation, the paste was sent to a third party service provider for purification (FIG. 10). Through two rounds of purification (FIG. 11), more than 40 milligrams of orientin and isoorientin with a purity of 95% were obtained, with a yield of over 40%. The above data was obtained through the conventional purification method adopted by service provider.

### SUMMARY

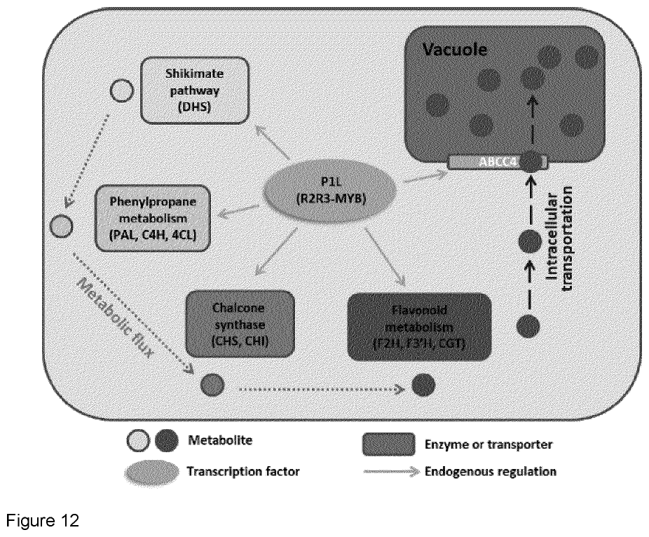

LtP1L not only acts as an transcriptional activator of early biosynthetic genes of flavonoid biosynthesis pathway, but also is responsible for the intracellular transport of flavonoids, rendering L. turionifera to accumulate a higher level of CGFs (FIG. 12).

### REFERENCES

Brazier-Hicks, M., Evans, K. M., Gershater, M. C., Puschmann, H., Steel, P. G., Edwards, R., 2009. The C-glycosylation of flavonoids in cereals. J. Biol. Chem. 284, 17926-17934.
Casas, M. I., Duarte, S., Doseff, A. I., Grotewold, E., 2014. Flavone-rich maize: an opportunity to improve the nutritional value of an important commodity crop. Front. Plant Sci. 5, 440.
Choi, J. S., Islam, M. N., Ali, M. Y., Kim, Y. M., Park, H. J., Sohn, H. S., Jung, H. A., 2014. The effects of C-glycosylation of luteolin on its antioxidant, anti-Alzheimer's disease, anti-diabetic, and anti-inflammatory activities. Arch. Pharm. Res. 37, 1354-1363.
Chu, S. S., Wang, J., Zhu, Y., Liu, S. L., Zhou, X. Q., Zhang, H. R., Wang, C. E., Yang, W. M., Tian, Z. X., Cheng, H., Yu, D. Y., 2017. An R2R3-type MYB transcription factor, GmMYB29, regulates isoflavone biosynthesis in soybean. PLoS. Genet. 13, e1006770.
Courts, F. L., Williamson, G., 2015. The occurrence, fate and biological activities of C-glycosyl flavonoids in the human diet. Crit Rev. Food Sci. Nutr. 55, 1352-1367.
Czemplik, M., Mierziak, J., Szopa, J., Kulma, A., 2016. Flavonoid C-glucosides derived from flax straw extracts reduce human breast cancer cell growth in vitro and induce apoptosis. Front. Pharmacol. 7, 282.
Du, Y. G., Chu, H., Chu, I. K., Lo, C., 2010. CYP93G2 is a flavanone 2-hydroxylase required for C-glycosylflavone biosynthesis in rice. Plant Physiol. 154, 324-333.
Ferreyra, M. L. F., Rodriguez, E., Casas, M. I., Labadie, G., Grotewold, E., Casati, P., 2013. Identification of a bifunctional maize C- and O-glucosyltransferase. J. Biol. Chem. 288, 31678-31688.
Ku, Y. S., Ng, M. S., Cheng, S. S., Lo, A. W. Y., Xiao, Z. X., Shin, T. S., Chung, G. H., Lam, H. M., 2020. Understanding the composition, biosynthesis, accumulation and transport of flavonoids in crops for the promotion of crops as healthy sources of flavonoids for human consumption. Nutrients 12, E1717.
Liu, J. Y., Osboum, A., Ma, P. D., 2015. MYB transcription factors as regulators of phenylpropanoid metabolism in plants. Mol. Plant 8, 689-708.
Martens, S., Preuss, A., Matern, U., 2010. Multifunctional flavonoid dioxygenases: Flavonols and anthocyanin biosynthesis in Arabidopsis thaliana L. Phytochemistry 71, 1040-1049.
Min, J. W., Hu, J. J., He, M., Sanchez, R. M., Huang, W. X., Liu, Y. Q., Bsoul, N. B., Han, S., Yin, J., Liu, W. H., 2015. Vitexin reduces hypoxia-ischemia neonatal brain injury by the inhibition of HIF-1 alpha in a rat pup model. Neuropharmacology 99, 38-50.
Morohashi, K., Casas, M. I., Ferreyra, L. F., Mejia-Guerra, M. K., Pourcel, L., Yilmaz, A., Feller, A., Carvalho, B., Emiliani, J., Rodriguez, E., Pellegrinet, S., McMullen, M., Casati, P., Grotewold, E., 2012. A genome-wide regulatory framework identifies maize Pericarp Color1 controlled genes. Plant Cell 24, 2745-2764.
Pagliuso, D., Jara, C. E. P., Grandis, A., Lam, E., Ferreira, M. J. P., Buckeridge, M. S., 2020. Flavonoids from duckweeds: potential applications in the human diet. RSC Adv. 10, 44981-44988.
Ren, D. B., Han, B. S., Xin, Z. Q., Liu, W. B., Ma, S. S., Liang, Y. Z., Yi, L. Z., 2016. Computation-aided separation of seven components from Spirodela polyrrhiza (L.) via counter-current chromatography. Sep. Purif. Technol. 165, 160-165.
Sasaki, N., Nishizaki, Y., Yamada, E., Tatsuzawa, F., Nakatsuka, T., Takahashi, H., Nishihara, M., 2015. Identification of the glucosyltransferase that mediates direct flavone C-glucosylation in Gentiana triflora. FEBS Lett. 589, 182-187.
Sonta, M., Rekiel, A., Batorska, M., 2019. Use of duckweed (Lemna L.) in sustainable livestock production and aquaculture—a review. Ann. Anim. Sci. 19, 257-271.
Stracke, R., Ishihara, H., Barsch, G. H. A., Mehrtens, F., Niehaus, K., Weisshaar, B., 2007. Differential regulation of closely related R2R3-MYB transcription factors controls flavonol accumulation in different parts of the Arabidopsis thaliana seedling. Plant J. 50, 660-677.
Sun, Y. W., Chen, Z., Yang, J. Y., Mutanda, I., Li, S. Y., Zhang, Q., Zhang, Y., Zhang, Y. L., Wang, Y., 2020. Pathway-specific enzymes from bamboo and crop leaves biosynthesize anti-nociceptive C-glycosylated flavones. Commun. Biol. 3: 110.
Vanegas, K. G., Larsen, A. B., Eichenberger, M., Fischer, D., Mortensen, U. H., Naesby, M., 2018. Indirect and direct routes to C-glycosylated flavones in Saccharomyces cerevisiae. Microb. Cell Fact. 17, 107.
   Yu, C. J., Sun, C. J., Yu, L., Zhu, M., Xu, H., Zhao, J. S., Ma, Y. B., Zhou, G. K., 2014. Comparative analysis of duckweed cultivation with sewage water and SH media for production of fuel ethanol. PLoS One 9, e115023.
Zhu, Q., Mao, L. N., Liu, C. P., Sun, Y. H., Jiang, B., Zhang, W., Li, J. X., 2016. Antinociceptive effects of vitexin in a mouse model of postoperative pain. Sci. Rep. 6, 19266.

Sequence Annex

The nucleotide sequence of LtP1-L is as follows (as shown in SEQ ID No.
1, with a direction of 5′-3′):
ATGGGGAGAGCGCCTTGCTGCGAGAAGGACGGGCTGAAGAAGGGGAGATGGACTGCGGAGGAAGAC
AAGATTCTGGCGAATTATATTCGAGTCAATGGAGAAGGTTCATGGAGATCGCTGCCGAAGAATGCA
GGACTTCTTCGATGCGGGAAGAGTTGCAGGCTGAGATGGATAAACTACCTCAGATCTGACTTGAAG
AGAGGCAACATTTCTCCGGAAGAGGAGGAGATGATCGTCCGGCTTCATACTTCTCTCGGAAACAGA
TGGTCTCTGATCGCCGCCCAGCTGCCTGGGAGGACGGATAACGAGATCAAGAACTACTGGAACTCC
CATCTGAGCCGTAGGATTCACCGGTTTAAGAGACTCGACGGGGGCGATGGCTCCACGGTGATCGTT
GACCTGAGCAAGATCGTCGGAATTCCGTGCCGGCGGCGAGGAGGAAGAAAAAAGATTTCGCCAATC
CAAAGCGTTGAGCCCGAGAGTAAAAAAGTCGACCACAAGGAGATGCTGATGGAGACGGCGACGAAC
GAGTTGCTGAGCCCGAGTTGGGAGAGCGGCGGGTCGATGAGCTCGTGTACGACGAGCGAGGAGAGG
AAAGACGAGCCAGTTCCGTCAAAGGAAAAACAGCTCTACGCTCATGGAGAAGAGTGCAACAGCTCT
GCAACAACGGAGGGAGAAATGCTTGACGTGGACTGGGAAGCCATGGCTACGAAGCTTTGGGATAAT
GACGAGATGGAGGAAATGTGGCCATGGCTCTGGGATAATAGCTCGCATGGATTCCAGCTCAAGTCT
CATCTGCAGCCGCCGGAGGGTGGGCAAGAGGAGACAGATCTCGGGGAATGGATCCTCTGA.
The amino acid sequence of LtP1-L is as follows (SEQ ID No. 2):
MGRAPCCEKDGLKKGRWTAEEDKILANYIRVNGEGSWRSLPKNAGLLRCGKSCRLRWINYLRSDLK
RGNISPEEEEMIVRLHTSLGNRWSLIAAQLPGRTDNEIKNYWNSHLSRRIHREKRLDGGDGSTVIV
DLSKIVGIPCRRRGGRKKISPIQSVEPESKKVDHKEMLMETATNELLSPSWESGGSMSSCTTSEER
KDEPVPSKEKQLYAHGEECNSSATTEGEMLDVDWEAMATKLWDNDEMEEMWPWLWDNSSHGFQLKS
HLQPPEGGQEETDLGEWIL.
The nucleotide sequence of LeP1-L is as follows (as shown in SEQ ID
No. 5, with a direction of 5′-3′):
ATGGGGAGGGCGCCGTGTTGTGAGAAAGTGGGAATGAAGAAGGGAAGATGGACGGCAGAGGAGGAC
GAGATACTGGCGAACTACATAAAGAAGAACGGAGAAGGTTCCTGGAGATCGTTGCCGAAGAAAGCA
GGTCTCTCGAGGTGCGGAAAGAGCTGCAGACTTCGCTGGATAAACTATCTGAGAAGTGATCTCAAG
AGGGGAAACATCACTTTGGAGGAAGAGGAGACCATCCTGAAGCTTCACACTTCTCTCGGCAACAGA
TGGTCCCTGATAGCCGCCCACCTTCCCGGAAGAACGGACAACGAGATAAAGAACCACTGGAACGCG
CACCTCAGCCGCCGAATCCACGTCTTCGGGCGGGGTGACCCCGTGATCCTGGATCTCGGACGGATG
GCTCACGGGAGACGGCGGGGGCATGAAGCCGCTGCCGAACCCACCTCCACGAAGAGACCAGGCGGC
GGCGGTGCCGCTGCGGTGAAAATCGCCGGACAAGAAGAGCTATTGGTAAGAGAGAAACCCCTGATC
GATGGGGGAATCTTGGAAGAGTTTGAGATCCTTCCCGGCGAGTTCTGGGAGATGATTGATGATATC
GGCGGGAATATGCTCTCCGCAAGCGCGACGTCCACCCCGGCGACGAGCGGGGAGGTTGACTCGACG
GGTGCTGGAACGGGAGACTTCGGCGAGCTCTCGTGCGGCAGCAGCGGTGGCAAGATGTTCGGCGAG
CAGTCGGATGAGGAGTGGGAAATCATGGCGGCACAGCTATGGGACGGGACCGAAAAAGCGTCGCCG
AGGCGATCGGACGATTTCATTTACGACGACGGCGCCCTCGACTGGTTCATCTCTCACGTCCTGTCG
TAA
The amino acid sequence of LeP1-L is as follows (SEQ ID No. 6):
MGRAPCCEKVGMKKGRWTAEEDEILANYIKKNGEGSWRSLPKKAGLSRCGKSCRLRWINYLRSDLK
RGNITLEEEETILKLHTSLGNRWSLIAAHLPGRTDNEIKNHWNAHLSRRIHVFGRGDPVILDLGRM
AHGRRRGHEAAAEPTSTKRPGGGGAAAVKIAGQEELLVREKPLIDGGILEEFEILPGEFWEMIDDI
GGNMLSASATSTPATSGEVDSTGAGTGDFGELSCGSSGGKMFGEQSDEEWEIMAAQLWDGTEKASP
RRSDDFIYDDGALDWFISHVLS

1. A method for producing a plant with modified flavonoid biosynthesis, the method comprising altering the expression, or activity, of a Duckweed Pericarp Color1 (P1) protein homologue in the plant.

2. A method as claimed in claim 1 for producing a plant with modified flavonoid biosynthesis, the method comprising altering the expression, or activity, of LtP1-L or LeP1L protein in the plant.

3. The method as claimed in claim 2 wherein the flavonoids are orientin and/or isoorientin.

4. The method as claimed in claim 2 or claim 3 wherein the method comprises increasing the expression of the LtP1-L or LeP1L protein in the plant.

5. The method as claimed in any one of claims 2 to 4 wherein the method:
(i) increases biosynthesis of phenylalanine and/or p-coumaroyl CoA; and/or
(ii) is performed in Lemna turionifera and activates the expression of LtABCC4 to modify the transport of orientin and/or isoorientin to the vacuole.

6. The method as claimed in any one of claims 2 to 5 wherein the method increases the content of total flavonoids in the plant by about 2, 2.5, or 3 times compared to the wild type plant.

7. The method as claimed in any one of claims 2 to 6 wherein the method increases the content of orientin and isoorientin by about 5, 6, 7, 8, 9, or 10 or more times compared to the wild type plant.

8. The method as claimed in any one of claims 2 to 7 the LtP1-L protein has a sequence with at least 70% identity to SEQ ID NO:2, more preferably has SEQ ID NO:2.

9. The method as claimed in any one of claims 2 to 8 wherein the LtP1-L protein comprises conserved R2 and R3 domains and/or the LtP1-L protein comprises a partial SG7-2 motif [K/R][R/x][R/K]xGR and a partial SG7 motif.

10. The method as claimed in any one of claims 2 to 9 wherein the LtP1-L protein is a polypeptide fragment of SEQ ID No. 2, preferably comprising at least 50 contiguous amino acids, more preferably at least 100 contiguous amino acids, more preferably at least 150 contiguous amino acids, more preferably at least 200 contiguous amino acids of SEQ ID No. 2.

11. The method as claimed in any one of claims 1 to 10 wherein LtP1-L protein is encoded by a LtP1-L polynucleotide which has at least 70% identity to SEQ ID NO: 1, more preferably which has SEQ ID NO: 1.

12. The method as claimed in any one of claims 2 to 7 the LeP1-L protein has a sequence with at least 70% identity to SEQ ID NO:6, more preferably has SEQ ID NO:6.

13. The method as claimed in any one of claims 2 to 7 or claim 12 wherein the LeP1-L protein comprises conserved R2 and R3 domains and/or the LtP1-L protein comprises no SG7-2 motif and a partial SG7 motif.

14. The method as claimed in any one of claims 2 to 7 or 12 to 13 wherein the LeP1-L protein is a polypeptide fragment of SEQ ID No. 6, preferably comprising at least 50 contiguous amino acids, more preferably at least 100 contiguous amino acids, more preferably at least 150 contiguous amino acids, more preferably at least 200 contiguous amino acids of SEQ ID No. 6.

15. The method as claimed in any one of claims 2 to 7 or 12 to 14 wherein LeP1-L protein is encoded by a LeP1-L polynucleotide which has at least 70% identity to SEQ ID NO: 5, more preferably which has SEQ ID NO: 5.

16. The method as claimed in any one of claims 2 to 15 wherein the plant is selected from a species of Rutaceae, Ginkgoaceae, Leguminosae, Lamiaceae and Compositae.

17. The method as claimed in any one of claims 2 to 15 wherein the plant is duckweed, preferably Lemna turionifera.

18. The method as claimed in claim 2 wherein LtP1-L polynucleotide is used to regulate biosynthesis of orientin and/or isoorientin in duckweed, wherein the nucleotide sequence of LtP1-L is shown in SEQ ID No. 1.

19. The method as claimed in claim 2 wherein LeP1-L polynucleotide is used to regulate biosynthesis of orientin and/or isoorientin in duckweed, wherein the nucleotide sequence of LeP1-L is shown in SEQ ID No. 5.

20. The method as claimed in claim 18 or claim 19 wherein overexpression of LtP1-L or LeP1-L polynucleotide is used to increase the content of orientin and/or isoorientin in duckweed.

21. The method as claimed in of claim 2 wherein LtP1-L protein is used to regulate the biosynthesis of orientin and/or isoorientin in duckweed, wherein the amino acid sequence of LtP1-L protein is shown in SEQ ID No. 2.

22. The method as claimed in of claim 2 wherein LeP1-L protein is used to regulate the biosynthesis of orientin and/or isoorientin in duckweed, wherein the amino acid sequence of LeP1-L protein is shown in SEQ ID No. 6.

23. The method as claimed in any one of claims 2 to 22 wherein the method comprises transforming the plant or a cell thereof to express the LtP1-L or LeP1-L protein in the plant or plant cell.

24. The method as claimed in claim 23 wherein the method comprises transforming the plant with an LtP1-L or LeP1-L polynucleotide encoding the LtP1-L or LeP1-L protein respectively, wherein the LtP1-L or LeP1-L polynucleotide is optionally operably linked to a heterologous promoter.

25. The method as claimed in any one of claims 18 to 24, which comprises the step of introducing the LtP1-L or LeP1-L polynucleotide into the duckweed genome thereby overexpressing LtP1-L or LeP1-L respectively such as to provide a higher content of orientin and/or isoorientin than in a corresponding wild-type plant.

26. The method as claimed in claim 25, wherein the introduction is mediated by Agrobacterium to infect duckweed callus tissue.

27. The method as claimed in any one of claims 2 to 21 wherein the method comprises modifying the sequence of an endogenous LtP1-L or LeP1-L polynucleotide encoding the LtP1-L or LeP1-L respectively protein in the plant or a cell thereof.

28. An isolated LtP1-L polynucleotide encoding an LtP1-L protein as defined in any one of claims 8 to 11.

29. An isolated LeP1-L polynucleotide encoding an LeP1-L protein as defined in any one of claims 12 to 15.

30. A genetic construct comprising the LtP1-L or LeP1-L polynucleotide of claim 28 or claim 29, optionally in which the polynucleotide sequence is operably linked to a heterologous promoter.

31. The genetic construct of claim 30 which is a plant expression vector containing the LtP1 or LeP1-L polynucleotide.

32. A cell comprising at least one of:
a) a heterologous polynucleotide of claim 28 or claim 29, or
b) a genetic construct of claim 30 or claim 31.

33. A method for producing a transgenic plant, which method comprises the steps of:
(a) performing a method as claimed in any one of claims 23 to 27 to produce a transformed plant cell, and
(b) regenerating a plant from the transformed plant cell.

34. A method for obtaining transgenic duckweed with high content of orientin and/or isoorientin, the method comprising:
(i) introducing LtP1-L polynucleotide encoding an LtP1-L protein as defined in any one of claims 8 to 11, or LeP1-L polynucleotide encoding an LeP1-L protein as defined in any one of claims 12 to 15, into the duckweed genome,
(ii) overexpressing LtP1-L or LeP1-L, and obtaining transgenic duckweed with higher content of orientin and/or isoorientin than wild-type plants.

35. A plant which:
a) comprises a plant cell of claim 32; and/or
b) is obtained or obtainable by the method of claim 33 or claim 34.

36. A part, propagule or progeny of a plant of claim 35, comprising the plant cell of claim 32.

37. Transgenic duckweed having a content of orientin and isoorientin of about 0.29% to 0.37%, or about 0.53% to 0.73% respectively, more preferably about 0.29% or 0.37%, or about 0.53% or 0.73% respectively.

38. A process of producing a flavonoid, which is optionally orientin and/or isoorientin, in a plant, which method comprises culturing or growing the plant of claim 35 or 37, and isolating the flavonoid from the plant.

39. A method for producing orientin and/or isoorientin, the method comprising:
(i) performing the method of claim 34;
(iii) cultivating the transgenic duckweed; and
(iv) extracting the orientin and/or isoorientin.

40. The method for producing orientin and/or isoorientin as claimed in claim 39, the method comprising: cultivating duckweed to obtain duckweed culture medium and extracting the orientin and/or isoorientin from the culture medium, optionally after removing the duckweed from the duckweed culture medium to obtain a culture aqueous solution, and extracting orientin and/or isoorientin from the culture aqueous solution.

41. The method for producing orientin and/or isoorientin as claimed in claim 39 or claim 40, wherein the duckweed is cultivated using an aqueous solution containing sucrose, optionally wherein the concentration of sucrose is about 1%, more preferably 2% g/ml.

42. The method for producing orientin and/or isoorientin as claimed in any one of claims 39 to 41, wherein the cultivation conditions are photoperiod: light 16 hours/dark 8 hours, 25° C.

43. A culture solution of transgenic duckweed having a content of orientin and isoorientin of about 16.83 mg/L to 22.38 mg/L and 24.89 mg/L to 30.54 mg/L respectively, more preferably about 16.83 mg or 22.38 mg/L, or about 24.89 mg/L or 30.54 mg/L respectively.

44. The culture solution of claim 43 which is a hydroponic solution.

45. Use of any of genetically engineered duckweed of claim 37, or obtained by the method of claim 34, or a part thereof or its culture medium, or orientin and/or isoorientin obtained from either in an industrial process, which is optionally in a food or feed product, a health product, or a cosmetic product.
